(19) United States
(12) Patent Application Publication
(10) Pub. No.: US 2007/0259029 A1
(43) Pub. Date: Nov. 8, 2007

(54) Water-dispersible patch containing an active agent for dermal delivery

(76) Inventors: Edward Enns McEntire, Kingsport, TN (US); Rebecca Reid Stockl, Kingsport, TN (US); Ramesh Chand Munjal, Kingsport, TN (US)

Correspondence Address: Tammye L. Taylor;Eastman Chemical Company, P.O. Box 511, Kingsport, TN, 37662-5075, US

(21) Appl. No.: 11/800,727

(22) Filed: May 7, 2007

Related U.S. Application Data
(60) Provisional application No. 60/798,575, filed on May 8, 2006.

(51) Int. Cl. A61K 9/70

(52) U.S. Cl. 424/449

(57) ABSTRACT

A dermal patch having comprising at least two layers wherein at least one layer is a polymer matrix system having an active agent admixed therein. At least one of the layers includes a water-dispersible or water-dissipatable polymer. The dermal patch has an elongation factor of at least 50%.

### CROSS REFERENCE TO RELATED APPLICATIONS

Benefit is claimed to the earlier filed application having U.S. Ser. No. 60/798,575, filed May 8, 2006 the entire disclosure of which is incorporated herein by reference.

### FIELD OF THE INVENTION

The present invention relates to a device for delivering an active agent to the epidermis or skin of a wearer. More particularly, the invention relates to a dermal patch having at least two layers wherein at least one of the layers includes an active agent.

### BACKGROUND OF THE INVENTION

Most skin or mucosal membrane diseases or disorders, such as eczema, psoriasis, dermatitis, as well as infections from bacteria, fungal, parasitic, allergic, hormonal and other environment agents produce a inflammatory response. One important route for the administration of one or more drugs, or other active agents for treating a skin or mucosal membrane is by topical application of the active agent onto the skin. The localized treatment of body tissues, diseases and wounds requires that the particular active ingredient or agent be maintained at the treatment site for an effective period of time.

Transdermal patches, which permit the controlled release of the active ingredients onto the skin, are well known. Two types of patches for skin applications are described in the literature. The first type of patches has a multi-layer structure where the active ingredients are dissolved or dispersed in the various layers. Multi-layer patches have a structure comprising several successive layers. For example such patches have a support layer, which is typically occlusive and can be composed of a material impermeable to the active compound to prevent its evaporation and to facilitate transdermal migration; a storage layer containing the active compound and capable of placement directly in contact with the skin; an adhesive layer applied to the surface of the storage layer and permeable to the active compound for attaching the patch to the skin; and a detachable protective layer to protect the laminated structure from any external contamination during storage prior to use of the patch.

Of increasing interest is the second type of patch where the active, such as a transdermal drug is dissolved or dispersed into a pressure sensitive adhesive layer, which serves not only to carry the bioactive substance but to also attach the patch to the skin. In the pressure sensitive adhesive patches, the bioactive substances are mixed with and formulated into a pressure sensitive adhesive matrix which may be subsequently coated as a single pressure sensitive adhesive layer which serves not only to carry the bioactive substance but to also to attach the patch to the skin. The adhesive matrix type dermal or transdermal patch is typically of a simple design having an impermeable, generally occlusive, backing layer, a single pressure sensitive adhesive, i.e., the adhesive matrix, and attached to the adhesive layer a removable release liner.

Prior art examples of patches for transdermal delivery of cosmetically active substances include U.S. Pat. No. 3,577,516 to Gould et al. discloses a spray-on bandage for protecting a wound to the skin comprising a gelling plastisol mixture of a polymer powder selected from polymers of hydroxy lower alkyl acrylates, methacrylates and copolymers of the same; and a high boiling polar plasticizer solvent. The patent further discloses that the plastisol can include a medically active ingredient which will diffuse from the film to the wound area over an extended period of time. The medically active ingredient can be incorporated into the film by impregnating the polymer with the agent; mixing the active agent with the polymer powder; or dissolving or dispersing the active ingredient in the high boiling plasticizer-solvent.

U.S. Pat. No. 5,626,866 to Ebert et al. discloses a transdermal or transmucosal drug delivery device having a drug-containing adhesive composite layer. The adhesive composite layer has first and second drug permeable adhesive layers containing a drug in gel form. The drug-containing adhesive composite layer is formed by extruding the drug, in gel form, onto at least one exposed surface of either the first or second adhesive layers and then joining the first and second adhesive layers together.

U.S. Pat. No. 5,965,154 to Haralambopoulos discloses an improved method for making an adhesive matrix type transdermal patch wherein ordinary, prefabricated, commercially available, pressure-sensitive adhesive tapes, with skin compatible adhesives, as the structural part of the patch is then loaded with the desirable bioactive substances and adjuvants. The bioactive substance can be a powder, liquid, or semi-liquid, e.g., a gel or an emulsion. In making the adhesive matrix type transdermal patch a thin layer of the bioactive substance is placed between the adhesive surface of a tape and its release liner (or its backing layer, for a transfer tape), and subjecting the assembly to moderate heat and/or pressure to laminate the assembly. As a result, the bioactive substance becomes incorporated into the adhesive matrix of the tape while the tape remains adhesive over its entire delivery surface.

U.S. Pat. No. 6,010,716 to Saunal et al. discloses a pharmaceutical composition for the transdermal administration of oestradiol, or of other medicinal substances, from a film formed on the skin. The pharmaceutical composition for transdermal administration comprises: 1) a polymeric release matrix capable of forming a flexible film after drying, chosen from cellulose polymers or copolymers or from vinylpyrrolidone/vinyl acetate copolymers; 2) an active principle, in particular oestradiol; 3) a promoter of transcutaneous absorption of the active principle; and 4) a physiologically acceptable non-aqueous solvent capable of dissolving the release matrix, the active principle and the transcutaneous absorption promoter and also capable of being rapidly removed by evaporation on contact with the skin.

U.S. Pat. No. 6,143,319 to Meconi et al. discloses a transdermal therapeutic system for the controlled release of estradiol or its pharmaceutically acceptable derivative alone consisting of a backing layer, an active-substance-containing reservoir which is produced by using pressure sensitive adhesives, and a removable protective layer. The pressure sensitive adhesive includes esters of colophony and inactive ingredients. The estradiol-containing pressure sensitive adhesive may additionally include polymers selected from the group consisting of styrene-butadiene-styrene block copolymers, styrene-isoprene-styrene block copolymers, styrene-ethylene-butylene-styrene block copolymers, ethylene-vinyl acetate copolymers, polyvinyl pyrrolidone, cellulose derivatives, and polymers based on acrylic acid and methacrylic acid derivatives.

U.S. Pat. No. 6,685,682 to Heinecke et al. discloses pressure sensitive adhesive composite dressing having a conformable backing with a pressure sensitive adhesive coated on a bottom face and an optional low adhesion coating on a top face, and which is supported by a removable carrier attached to the top face of the backing. The carrier is nonpermanently heat-sealed to the backing and a cut in the carrier substantially defining a window proximate a center of the carrier, the carrier further being formed of material substantially more rigid than the backing to provide rigidity to the adhesive composite.

U.S. Patent Application Publication No. 2003/0152612 to Pugliese et al. discloses a multi-layered, transdermal patch. The patch must have at least two layers: the first layer comprises a pressure-sensitive, adhesive layer containing an adequate amount of a selected xanthine, uniformly distributed therein, for delivery to the skin over the duration of the patch application; the second layer consists of a backing film or a fabric, which provides protection to the xanthine containing adhesive first layer when applied to skin. The patch can be applied to the desired skin site by means of the adhesive first layer, which tightly adheres to the skin. Then, xanthine from the patch continuously diffuses through the skin into the underlying tissues including the fatty tissues. The xanthine-containing, adhesive may be selected from an acrylate copolymer, a vinyl ether polymer, or a silicone adhesive polymer.

U.S. Patent Application Publication No. 2003/0157138 to Eini et al. discloses a pharmaceutical or cosmetic carrier comprising 1-25 weight % of a solidifying agent and 75-99 weight % of a hydrophobic solvent, and the solvent is typically liquid at ambient temperature. The solidifying agent includes at least one long chain fatty alcohol having at least 15 carbon atoms in its carbon backbone and/or at least one fatty acid, having at least 18 carbon atoms in its carbon backbone. The hydrophobic solvent includes at least one marine animal derived oil, at least one terrestrial animal derived oil, at least one mineral oil, at least one silicone oil and/or at least one plant-derived oil.

U.S. Patent Application Publication No. 2003/0175333 to Shefer et al. discloses an invisible patch for the controlled delivery of cosmetic, dermatological, and pharmaceutical active ingredients onto the skin formed of a single matrix layer. The patch is a single layer water soluble matrix comprising one or more water sensitive bioadhesive polymers, a water soluble oligomer, and a surfactant. Upon application onto a wetted skin surface, the patch dissolves or disintegrates and provides a substantive therapeutic layer to the treatment site over an extended period of time.

U.S. Patent Application Publication No. 2004/0033254 to Song et al. discloses an active agent containing adhesive composition for the transdermal delivery of hydrophilic drugs. The matrix layer is made up of outer adhesive layers comprising an active ingredient, a hydrophobic acrylic adhesive polymer, a mixture of high and low molecular weight water soluble polyvinylpyrrolidone (PVP) polymers and colloidal silica. Sandwiched between the adhesive layers is an absorption enhancing layer comprising ingredients selected from the group consisting of non-ionic surfactants, terpenes and dissolution assisting agents.

U.S. Patent Application Publication No. 2004/0127531 to Lu et al. discloses a pharmaceutical composition having a flexible backing sheet with opposing surfaces that are distal and proximal to the skin when applied and a coating on the proximal surface of the backing sheet. The coating comprises (a) an adhesive and (b) an active agent comprising valdecoxib or a prodrug thereof being in a therapeutically effective total amount and dispersed in a matrix that comprises zero to less than an active agent solubilizing effective amount in total of one or more solvents other than the adhesive.

Many groups of drugs intended for topical application, such as antibiotics, antifungal, anti-inflammatory, anesthetic typically include a hydrophobic carrier such as petrolatum, liquid paraffin, lanolin, beeswax, vegetable oil, glycerin monostearate, polyethylene glycol and some emulsifying agents have limited use due to their consistency and greasy feeling following topical application. Another problem is if a patch having a hydrophobic carrier is used over an extended period of time without removal the hydrophobic carriers may interfere with moisture evaporation from the skin leading to skin maceration.

Additionally, several hydrophobic liquids, such as mono- and polyunsaturated oils from vegetable and marine sources, silicone oils, mineral oils, and liquid hydrophobic plant-derived oils are known for their therapeutic effects when applied topically. The oils may also contain essential nutritional constituents, such as oil soluble vitamins, minerals and other therapeutically effective constituents. However, administration of such therapeutic oils in a liquid form does not exert sufficient amounts of the therapeutic oils due to their flow-spread properties. A problem with incorporating such hydrophobic carriers and/or liquids is that they may interfere with the tackiness of the adhesive, resulting in a patch that does not stick to the intended site or the patch is easily dislodged.

A further disadvantage of the patches of the prior art is that they are typically multiple layer devices that are thick, readily visible to others, and have a backing layer that although flexible is not sufficiently stretchable to allow for freedom of movement.

Accordingly, there is a need in the art for a topically applied, layered composition or patch having a pharmaceutical, cosmetic or dermatological agent included. There is a further need for such type of patch that is relatively thin, having a substantially non-tacky exposed surface and that would be flexible, stretchable and substantially non-perceptible during use.

### BRIEF SUMMARY OF THE INVENTION

Briefly, the present invention is a dermal patch having at least two wherein at least one of the layers is a polymer matrix system having an active agent admixed therein, wherein at least one of the layers is water dispersible and wherein the dermal patch has an elongation factor of at least 50%.

One aspect of the invention pertains to a dermal patch having a first and a second layer, wherein at least one of said layers is a polymer matrix system having an active agent admixed therein, wherein at least one layer comprises a water-dispersible or water-dissipatable film-forming polymer, and wherein the dermal patch has an elongation factor of at least 50%.

Another aspect of the invention pertains to a dermal patch having a first layer and a second layer, wherein at least one layer comprises from about 25 to about 99.8 weight % of a water-dispersible or water-dissipatable film-forming polymer and at least one layer comprises from about 0.1 to about 50 weight % of an active agent, wherein the sum of the ingredients in each layer equals 100 weight %.

Yet another aspect of the present invention is a method for making the dermal patch of the present invention. The method comprises the steps of combining a predetermined amount of the active agent with a predetermined amount of an acceptable water-dispersible or water-dissipatable polymer in an aqueous phase to form a blend, coating the blend on a suitable releasable substrate to form a first layer, drying the first layer, joining a second layer over the first layer and covering the second layer with a suitable releasable substrate.

### BRIEF DESCRIPTION OF THE DRAWINGS

FIG. 1 is a cross-sectional view of one embodiment of the dermal patch of the present invention.

FIG. 2 is a cross-sectional view of the dermal patch of the present invention shown as applied during use.

FIG. 3 is a cross-sectional view of another embodiment of the dermal patch of the present invention having illustrating the top layer as an intermittent layer.

FIG. 4 is a cross-sectional view of another embodiment of the dermal patch of the present invention having illustrating the bottom layer as an intermittent layer.

FIG. 5 is a perspective view of the embodiment of FIG. 3 shown as applied during use.

### DETAILED DESCRIPTION OF THE INVENTION

The present invention may be understood more readily by reference to the following detailed description of the invention, and to the Examples included therein.

Before the present compositions of matter and methods are disclosed and described, it is to be understood that this invention is not limited to specific methods or to particular formulations, unless otherwise indicated, and, as such, may vary from the disclosure. It is also to be understood that the terminology used is for the purpose of describing particular embodiments only, and is not intended to limit the scope of the invention.

The singular forms “a,” “an,” and “the” include plural referents, unless the context clearly dictates otherwise.

Optional or optionally means that the subsequently described event or circumstances may or may not occur. The description includes instances where the event or circumstance occurs and instances where it does not occur.

Ranges may be expressed herein as from about one particular value, and/or to about another particular value. When such a range is expressed, it is to be understood that another embodiment is from the one particular value and/or to the other particular value.

Where patents or publications are referenced, the disclosures of these references in their entireties are intended to be incorporated by reference, in order to more fully describe the state of the art to which the invention pertains.

The dermal patch of the present invention provides an effective residence time with minimal discomfort and ease of use, and is an appropriate vehicle for local as well as systemic delivery of cosmetic, dermatological, and pharmaceutical active ingredients to the surface of skin or to the surrounding tissues. One skilled in the art will understand that the patch of the present invention can have any dimension that would be convenient to the user or for delivery of any cosmetic, dermatological, and pharmaceutical active ingredient.

As used herein, the term “matrix”, “polymer matrix” and “adhesive matrix” are used interchangeably depending upon their context to mean a cosmetic, dermatological, or pharmaceutical active ingredient intimately admixed, dissolved or suspended in a biocompatible polymeric phase. As used herein, the term “adhesive matrix” is more specific and means a cosmetic, dermatological, or pharmaceutical active ingredient intimately admixed, dissolved or suspended in a biocompatible polymeric phase wherein the polymeric matrix is adapted to have pressure sensitive adhesive characteristics. One skilled in the art will understand that the matrix can also contain other ingredients. This definition is meant to include embodiments wherein such polymeric phase is laminated to a pressure sensitive adhesive or used with an overlay adhesive.

In accordance with the present invention, a dermal patch is provided having at least two layers. At least one of the layers contains an active ingredient that may be fixed within the layer or that may be fugitive from the layer. At least one of the layers comprises a water-dispersible or water-dissipatable polymer. Advantageously, the dermal patch of the present invention also has an elongation factor of at least 50%.

As used herein, the term “patch” is a generic term which is to be understood as covering any type of known devices, such as swatches, tapes poultices, pads, plasters, cataplasms, and dressings that are adhesive to the skin.

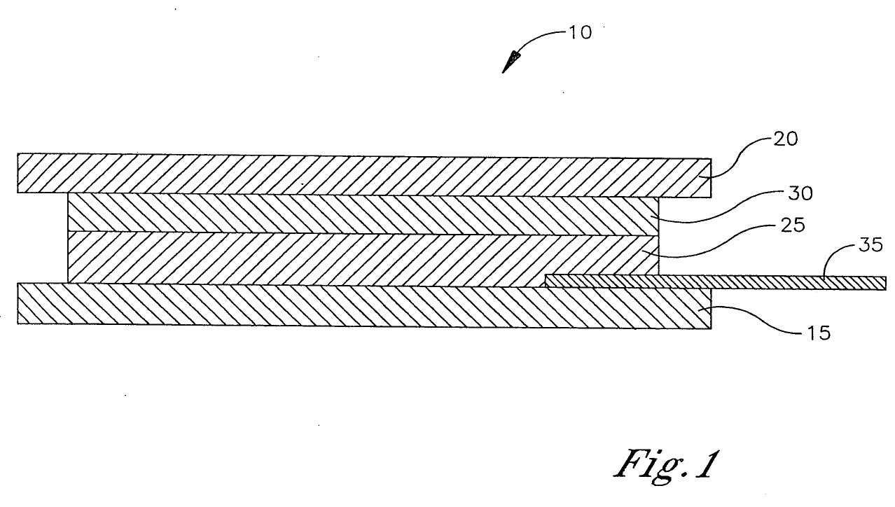

Referring to FIG. 1, an illustrative embodiment, of the dermal patch of the present invention is shown generally as 10. The dermal patch 10 shall be described with reference to the various components as would be applied to a user's skin. The dermal patch 10 has a first protective covering 15 and a second protective covering 20, overlaying the first and second layers 25 and 30, respectively. First and second layers 25 and 30 are positioned between, but not coextensive with, the first and second protective coverings 15 and 20 to prevent contamination of the surfaces and to retain the hydrophilic nature of at least one of the layers.

The protective coverings 15 and 20, or release substrates, are desirably flexible, and easily separated from the respective layers 25 and 30. The protective covering 15 can be relatively more rigid than, as flexible as, or more flexible than the polymeric layer 25. The only criteria for the protective covering 15 is that it be readily removable for exposing the first layer 25, and that the protective covering 20 should be flexible enough to readily conform to the wear's skin.

Any suitable material can be used for the backing sheet. Typically the protective coverings 15 and 20 are a siliconized polymer film, such as polyethylene, polypropylene, polyvinyl chloride, ethyl vinyl acetate, polyurethane, polyester, oriented polyester, oriented polypropylene, silicone or wax treated paper, a woven or non-woven fabric which may optionally have one or more of the aforementioned polymers laminated onto a surface of the fabric. The fabric may also be siliconized, or treated with silicone release agent as is known to those skilled in the art. The protective coverings 15 and 20 may be impermeable to air and/or water. Preferably, the protective coverings 15 and 20 are an elastic film that is water-resistant and skin-conformable. A preferred protective covering material is a release film (Polyester Liner L-25X available from Sil-Tech, 222 Mound Avenue, Miamisburg, Ohio 45342).

Attached to a surface of the protective covering 15 is a grasping means 35 for separating the protective covering 15 from layer 25. The grasping means 35 is illustrated as a separate member affixed to the inner surface of the protective covering 15. However, one skilled in the art will understand that the grasping means 35 could be attached to the outer surface of the protective covering 15 as well as being an integral part of the protective covering 15. The manner by which grasping means 35 is affixed, i.e., an integral or separate member, its configuration, or manner of manufacture may vary according to the present invention. It is important, but not critical to the present invention, that the grasping means 35 provide a means by which the user can readily identify which side of the patch 10 is to placed adjacent to the wearer's skin and that the grasping means 35 facilitate removing the first protective covering 15 to expose the adhesive first layer 25 for dermal contact.

As illustrated, the grasping means 35 is a separate piece of adhesive tape applied to the protective covering 15 so that its adhesive side of the tape bonds with the adhesive release substrate 15 and is typically applied on at least one edge of the patch. Suitably, this tape can be many different types of adhesive tape, such as Scotch brand matte finish household or office tape available from 3M Company, Scotch Magic® Tape, Crystal Clear Duck tape available from Henkel Consumer Adhesives, Inc., electrical tape, or any other adhesive tapes which are thin films (about 0.5 to about 5 mils in thickness) of plastics such as polyethylene terephthalate or cellulose acetate that contain a strong adhesive. A preferred width is from about 0.5 to about 1 inch.

For ease of description, the adhesive or adhesive matrix, i.e., adhesive containing one or more active agents or other ingredients, is illustrated as being positioned adjacent to the protective covering 15. To apply the patch 10 to the skin, the user separates the protective covering 15 from the adhesive layer 25. The adhesive layer 25 is then pressed onto the skin, typically using the hand to apply pressure on the release layer 20. Once the adhesive layer 25 is firmly attached to the skin, the release layer 20 is grasped and peeled from the composite film. The adhesive layer 25 has a bonding strength to the second layer 30 and to the skin that is greater than the bonding strength of the protective layer 20 to the surface of the second layer 30. Thus, the second protective layer 20 is removed exposing the outer surface 40 of the second layer 30.

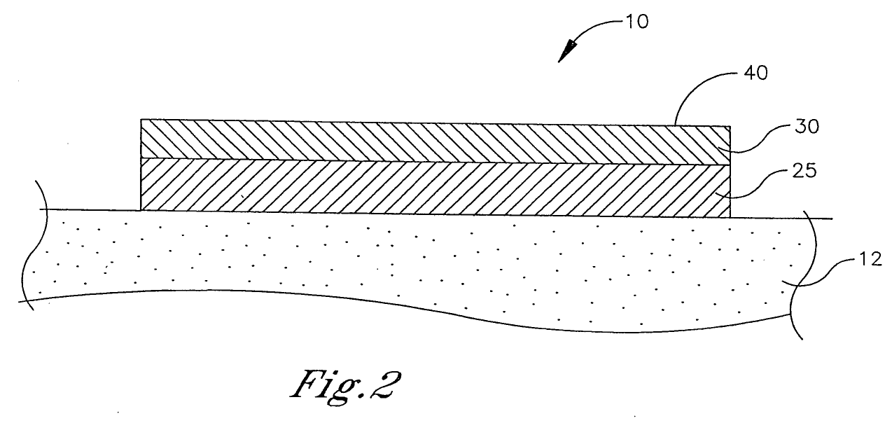

Referring to FIG. 2, a dermal patch 10 of the present invention is illustrated as applied to a wearer's skin 12. The dermal patch has at least two layers, wherein at least one of the layers is a polymer matrix system having an active agent admixed therein, and wherein at least one of the layers is water-dispersible, which may be the same or different from the matrix layer. It is also important aspect of the present invention that the dermal patch has an elongation factor of at least 50% so that the patch will move in conjunction with the wear's movements.

In the description that follows, all weight percentages are based on the total weight of the constituents comprising an individual layer, unless specifically designated as being directed to the total weight of the dermal patch.

The Adhesive Matrix Layer

The adhesive layer 25 is adapted to be positioned adjacent to the user's skin and has a sufficiently low glass transition temperature, Tg, to substantially adhere to the skin when applied. For ease of description of the present invention, the adhesive layer 25 is an adhesive matrix containing at least one active agent. The adhesive matrix 25 includes a film-forming, biocompatible polymer suitable for contact with the skin and an active ingredient. The adhesive matrix comprises: a) from about 25 to about 99.8 weight % of a film-forming, adhesive polymer; and b) from about 0.1 to about 50 weight % of an active ingredient. Optionally, the adhesive matrix may include one or more of c) from about 0.1 to about 25 weight % of a surfactant; or d) less than about 10 weight % of a skin permeation enhancing agent; or e) up to about 20 weight % of a humectant; or f) up to about 20 weight % of a plasticizer, wherein the sum of the ingredients equals 100 weight %.

In another embodiment, the adhesive matrix includes a film-forming, biocompatible polymer suitable for contact with the skin and an active ingredient. The adhesive matrix comprises: a) from about 30 to about 95 weight % of a film-forming, adhesive polymer; and b) from about 1 to about 40 weight % of an active ingredient. Optionally, the adhesive matrix may further include one or more of c) from about 0.1 to about 25 weight % of a surfactant; or d) less than about 10 weight % of a skin permeation enhancing agent; or e) up to about 20 weight % of a humectant; or f) up to about 20 weight % of a plasticizer, wherein the sum of the ingredients equals 100 weight %.

In another embodiment, the adhesive matrix of the present invention comprises: a) from about 40 to about 95 weight % of a film-forming, adhesive polymer; and b) from about 1 to about 40 weight % of an active ingredient. Optionally, the adhesive matrix can further include c) from about 0.1-25 weight % of a surfactant; and/or d) less than about 10 weight % of a skin permeation enhancing agent; and/or e) up to about 20 weight % of a humectant; and/or f) up to about 20 weight % of a plasticizer, wherein the sum of the ingredients equals 100 weight %.

The polymers suitable for use as an adhesive matrix include uncrosslinked or crosslinked, film-forming, water-dispersible polymers. The polymer should have some compatibility with the active agent such that the desired amount of active agent can be incorporated into the polymer matrix without substantially affecting the adhesive quality of the polymer. Desirably, the polymer should not be absorbable into the skin. Polymers which may be used include water-dispersible or water-dissipatable polyester or polyester amides such as sulfopolyesters or polyesteramides (collectively referred to as polyester(s) or sulfopolyester(s)) containing ether groups and sulfonate groups having a glycol residue and a dicarboxylic acid residue and at least one difunctional comonomer containing a sulfonate group attached to an aromatic nucleus and in the form of a metallic salt. Such polymers are well known to those skilled in the art and are available from Eastman Chemical Company under the trade name of Eastman AQ polyester polymers. In particular, such sulfopolyesters can be dissolved, dispersed or otherwise dissipated in aqueous dispersions, preferably at temperatures of less than about 80° C. Such polyesters are described in greater detail in U.S. Pat. No. 3,734,874, issued to Charles Kibler on May 22, 1973, the disclosure of which is incorporated herein by reference. One skilled in the art will understand that the term “residue” or “component” as used in the specification and concluding claims, refers to the moiety that is the resulting product of the chemical species in a particular reaction scheme or subsequent formulation or chemical product, regardless of whether the moiety is actually obtained from the chemical species. Thus, for example, an ethylene glycol residue in a polyester refers to one or more —OCH₂CH₂O— repeat units in the polyester, regardless of whether ethylene glycol is used to prepare the polyester. The use of the term “acid” in the above description and in the appended claims includes the various ester forming or condensable derivatives of the acid reactants such as the dimethyl esters thereof as employed in the preparations set out in these patents. Among the preferred sulfo-monomers are those wherein the sulfonate group is attached to an aromatic nucleus such as benzene, naphthalene, diphenyl, or the like, or wherein the nucleus is cycloaliphatic such as in 1,4-cyclohexanedicarboxylic acid.

Another a suitable polymer for use in the present invention can be a hybrid latex of a sulfopolyester and acrylic as described in U.S. Pat. No. 6,001,922. Other examples of such sulfopolyester-acrylic hybrid polymers, wherein the acrylic monomers are polymerized in the presence of the sulfopolyester dispersion are found in U.S. Pat. No. 4,946,932, the entire disclosures of which is incorporated herein by reference.

Other polymers suitable for use in the current invention are sulfonated or sulfated acrylic copolymers prepared from acrylamide or acrylic type monomers such as 2-acrylamido-2-methyl propanesulphonic acid (AMPS®) available from Lubrizol or sulfoethyl methacrylate (SEM) available from Polysciences, Inc. The AMPS or SEM may be polymerized with other monomers such as methyl methacrylate, butyl acrylate, styrene, and the like to form acrylic polymers. The AMPS or SEM may be present in the polymer as a salt with ammonia, an amine, or an alkali metal.

Other polymers suitable for use in the present invention are water-dispersible acrylic polymers prepared using a miniemulsion process. Such miniemulsion processes are known to those skilled in the art. In the instant invention the term “miniemulsion” refers to a polymerization of small (generally <1000 nanometers) droplets of acrylic monomers in contrast to a conventional acrylic monomer polymerization wherein monomer droplet sizes present are in the range of about 1,000 to about 10,000 nanometers. The process is directed to forming hydrophobically modified emulsion polymers including a hydrophobic core and a hydrophilic shell. To stabilize the miniemulsion pre-emulsion (before polymerization) an organic hydrophobe is added. This organic hydrophobe may be an active ingredient such as defined herein, or may be added in addition to the active ingredient.

Other polymers are polymers known as sulfonate stabilized water dispersible acrylic polymers are available from ALCO Chemical Company, Chattanooga, Tenn. For example, sulfonated polystyrene polymers such as VERSA-TL Sulfonated Polymers are suitable. Furthermore, partially sulfonated polystyrene polymers neutralized as alkali metal salts are suitable. These may be used alone or in conjunction with other polymers. Other such water dispersible polymers are sulfonated polystyrene polymers such as those available from National Starch under the trade name FLEXAN® II.

Other suitable polymers include water dispersed polyurethane polymers. Suitable examples are polymers known as Avalure UR405 and Avalure UR450 available from Noveon Chemical Company.

In another embodiment the polymer forming the adhesive matrix layer comprises a sulfonated or sulfated acrylic polymer or sulfonated polyester. The acrylic or polyester polymer comprises water dispersing or water dissipating polar moieties such as sulfate or sulfonate, carboxylate, or polyethylene oxide. The adhesive matrix layer may contain blends of polymers of low Tg (below zero degrees Centigrade), with polymers of high Tg (above zero degrees Centigrade), and optionally a humectant, a plasticizer, a surfactant, a skin permeation enhancing agent, or a tackifier.

In another embodiment, the polymer forming the adhesive matrix layer comprises an acrylic polymer having glass transition temperature (Tg) less than about 0° C., such as, for example, less than about −5° C., or even less than about −10° C., whereupon the active ingredient is incorporated into the adhesive polymer layer. The term “acrylic polymer” is used interchangeably with “polyacrylate,” “polyacrylic polymer,” and “acrylic adhesive.” The acrylate polymers useful in practicing the invention are polymers of one or more monomers of acrylic acids and other copolymerizable monomers. The acrylate polymers also include copolymers of alkyl acrylates and/or methacrylates and/or copolymerizable secondary monomers or monomers with functional groups. By varying the amount of each type of monomer added, the cohesive properties of the resulting acrylic polymer can be changed, as is known in the art. In general, the acrylic polymer is composed of at least about 60 mole % of an acrylate or alkyl acrylate monomer and can contain up to about 40 mole % of a functional monomer copolymerizable with the acrylate, wherein the above mole percentages are based on the total mole of polymer comprising the film-forming acrylic pressure sensitive adhesive.

Examples of suitable acrylic monomers include, but are not limited to, styrenic monomers such as styrene, alpha-methyl styrene, vinyl naphthalene, vinyl toluene, and chloromethyl styrene; ethylenically unsaturated species such as, (meth)acrylic acids and esters having carbon chain lengths of up to about 30 carbon atoms, for example, methyl acrylate, acrylic acid, methacrylic acid, methyl methacrylate, ethyl acrylate, ethyl methacrylate, butyl acrylate, butyl methacrylate, isobutyl acrylate, isobutyl methacrylate, hexyl acrylate, hexyl methacrylate, ethylhexyl acrylate, ethylhexyl methacrylate, octyl acrylate, octyl methacrylate, fluoro or silicon containing monomers such as but not limited to octafluoropenta acrylate and trimethylsiloxyethyl acrylate, decyl acrylate, decyl methacrylate, dodecyl acrylate, dodecyl methacrylate, tridecyl acrylate, and tridecyl methacrylate, stearyl acrylate, cetyl acrylate, and the like. In addition, functional monomers such as hydroxyethyl acrylate, hydroxyethyl methacrylate, hydroxypropyl acrylate, glycidyl methacrylate, carbodiimide methacrylates such as cyclohexylcarbodiimidoethyl methacrylate; t-butylcarbodiimidoethyl methacrylate, and alkyl crotonates. Also suitable are vinyl acetate, vinyl neodecanoate, ethylene, propylene, butylene, butadiene, isoprene, di-n-butyl maleate, and di-octylmaleate; vinyl ethers such as methyl vinyl ether, butyl vinyl ether, cyclohexyl vinyl ether, sodium styrene sulfonate, sodium vinyl sulfonate, 2-acrylamido-2-methylpropane sulfonic acid or its salts, 2-sulfoethyl methacrylate or its salts; and nitrogen containing monomers including acrylonitrile, methacrylonitrile, acrylamide, methyl acrylamide, N,N-dimethyl acrylamide, methacrylamide, t-butylaminoethyl methacrylate, dimethylaminoethyl methacrylate, diethylaminoethyl methacrylate, N,N-dimethylaminopropyl methacrylamide, 2-t-butylaminoethyl methacrylate, N,N-dimethylaminoethyl acrylate, N-(methacryloyloxy-ethyl)ethylene urea and methacrlamidoethylethylene urea and mixtures thereof.

The acrylic moiety also may include any pendant moiety which is capable of (i) surviving the polymerization process and/or (ii) participating in or promoting crosslinking of the resin. An acrylic moiety may have an ethylenic unsaturation such as, but not limited to, allyl and vinyl groups. This group may also be an acetoacetoxy moiety or enamine moiety. Examples of acrylic monomers having latent oxidatively-functional groups include, but are not limited to, allyl methacrylate, vinyl methacrylate, acetoacetoxyethyl methacrylate, hydroxybutenyl methacrylate, the allyl or diallyl ester of maleic acid, and poly(allyl glycidyl ether).

As used herein the term “allyl” means a three carbon-carbon chain which includes one ethylenically unsaturated moiety having from 2 to 24 carbon atoms.

As used herein the term “alkyl” means branched, linear, and cyclic substitute carbon chain containing from one to about 30 carbon atoms.

Advantageously, the adhesive matrix layer may contain a high concentration of the active ingredient(s) contained therein which helps increase transdermal absorption efficiency and results in superior adhesion for long-term application onto the skin. Moreover, if the adhesive layer contains the active ingredient, the drying process of the adhesive layer can be achieved in a short period of time, which in turn significantly reduces the manufacturing time and cost. The total thickness of the patch of the present invention, excluding the backing layers, is from about 0.2 to about 5 mils (about 5 to about 125 micrometers), such as, for example, from about 0.5 to about 4 mils (about 12.5 to about 100 micrometers), or from about 0.7 to about 3.5 mils.

The water-dispersible or water dissipatable acrylic polymer matrix of the present invention may be prepared using a miniemulsion technique, whereby the active ingredient is present during polymer formation. In the instant invention the term “miniemulsion” refers to a polymerization of small (on average, generally ≦1000 nanometers in diameter) droplets of acrylic monomers in contrast to a conventional acrylic monomer polymerization wherein monomer droplet sizes present are in the range of about 1,000 to about 10,000 nanometers. The process is directed to forming modified emulsion polymers including a hydrophobic core and a hydrophilic shell. To stabilize the miniemulsion pre-emulsion (before polymerization) an organic hydrophobe is added. This organic hydrophobe may be an active ingredient such as defined herein, or may be added in addition to the active ingredient. In accordance with the method embodiment of the present invention, oils-acrylic hybrid emulsions may be prepared by incorporating hydrophobic, substantially saturated oils (such as coconut oil fatty acid or coconut oil) or unsaturated oils (such as natural oils such as avocado oil, Shea butter, and the like) in acrylic monomers. Accordingly, a water-based latex is prepared by polymerizing an acrylic monomer in the presence of hydrophobes. Depending upon the glass transition temperature (Tg) of acrylic portion of the hybrid-polymer, these latex emulsions can prove to be excellent film formers. Generally, colloidal polymer dispersions of the present invention contain from about 20 to about 60% polymer particles dispersed in water.

The average particle size of the modified acrylic latex may range from about 25 to about 500 nm. In another embodiment, particle sizes range from about 50 to about 300 nm, and in yet another embodiment particle sizes range from about 70 to about 250 nm. The latex particles generally have a spherical shape. Mini-emulsion polymerization can be accomplished by redox or thermal processes in batch, semi-continuous and continuous fashion.

The Second Layer

The second layer 30, may be the same as or different from the first layer 25. Although it is desirable that the second layer 30 include a water-dispersible or water-dissipatable polymer, it is not necessary if the first layer 25 is water-dispersible or water-dissipatable. In any manner, the second layer 30 should be compatible with and relatively strongly affixed to the first layer 25 and permit the patch 10 to have at least 50% elongation when used. Accordingly, the second layer 30 may be comprised of any polymer known to those skilled in the art, although a water-dispersible or water-dissipatable of the type described above is preferred. The second layer 30 should have a Tg such that it is substantially non-tacky or preferably tack-free in less than 3 minutes after the protective covering 20 is removed and the dermal patch is being used. As used herein the term “substantially non-tacky” means the film layer 30 no longer acquires fibers from a cofton ball (having a weight of 0.6 to 0.8 grams) being slowly rolled across the width of the film. This time corresponds approximately to the time at which the film, when pushed gently with a clean finger, no longer pulls on the finger as the finger was withdrawn.

Desirably, the glass transition temperature, Tg, of the polymer in the adhesive first layer 25 will be from about −5° C. to about −45° C., and the Tg of the polymer in the second layer 30 will be greater than about 5° C., wherein the delta or difference in Tg of the two polymers is at least about 15° C. In another embodiment, the delta or difference in Tg of the two polymers is at least about 25° C.

One skilled in the art will understand that to impart the desired Tg to either layer, it may be necessary to add a plasticizer that is both compatible with the polymer used in one or both of the layers as well as being suitable for dermal contact. Such plasticizers are well known to those skilled in the dermal patch art. Such plasticizers may be included in the polymer in an amounts up to about 20 weight % of the respective layer composition. In another embodiment the amount of plasticizer is from about 0 to about 25 weight % of the layer composition. Non-limiting examples of such plasticizers include diols, triols, polyols, alcohol ethers, alcohol esters, esters, ethers, carboxylic acids, hydroxy acids, amides, carbonates, and mixtures thereof. Suitable plasticizers include triacetin, triethyl citrate, glycerin, sorbitol, 1,2-propylene glycol, ethylene glycol, 1,3 propylene glycol, 2-methyl-1,3-propanediol, butylene glycol, hexylene glycol, isoprene glycol, xylitol, fructose, hexanediol, octanediol, and mixtures thereof.

To impart an imperceptibility to the film, a particulate material may be included in layer 30 to reduce the gloss. The particulate material may be inorganic or organic and may be added to the upper surface 40 (FIG. 2) of the film. The particle diameter may be from about 1 to about 10 micrometers, preferably from about 2 to about 5 micrometers. From about 1 to about 10 percent by weight of the particulate material may be added, based on the total weight of the patch 10. Preferably from about 2 to about 7 percent of the particulate material is added to the film. Examples are glass spheres, hollow glass spheres, ceramic spheres, silica spheres, alumina particles, polymer particles produced by grinding, and the like. Alternatively, to remove gloss the upper film's surface (away from the skin) may be embossed with a pattern to inhibit reflection.

The active ingredient useful in the practice of the present invention is selected from one or more cosmetic, dermatological, and pharmaceutical active ingredients that may be fixed within the incorporating layer or fugitive, i.e., migrates onto the skin surface or is absorbed into the skin. Depending upon a predetermined use or benefit to be derived from the active agent, the dermal patch of the present invention may have a fugitive active ingredient that transfers from about 1 to 100 weight % of the active agent to the epidermis of the user, preferably from about 10 to 100 weight % and more preferably greater than about 80 weight % of the fugitive active ingredient is transferred to the epidermis of the user.

Desirably, fugitive active agents have a beneficial effect on the skin, including, but not limited to: anti-oxidants; free radical scavengers; skin moisturizers; de-pigmentation agents; reflectants; humectants; antimicrobial (e.g., antibacterial) agents; allergy inhibitors; anti-acne agents; anti-aging agents; anti-wrinkling agents, antiseptics; analgesics; antitussives; antipruritics; local anesthetics; hair growth promoting agents, antihistamines; keratolytic agents; anti-inflammatory agents; fresheners; healing agents; anti-infectives; inflammation inhibitors; anticholinergics; vasoconstrictors; vasodilators; wound healing promoters; peptides, polypeptides and proteins; deodorants and antiperspirants; skin emollients; tanning agents; skin lightening agents; antifungals such as antifungals for foot preparations; depilating agents; external analgesics; counterirritants; insecticides; poison ivy products; poison oak products; burn products; anti-diaper rash agents; prickly heat agents; make-up preparations; vitamins; amino acids and their derivatives; herbal extracts; retinoids; flavoids; sensory markers (i.e., cooling agents, heating agents, etc.); skin conditioners; anti-cellulite agents; chelating agents; cell turnover enhancers; coloring agents; sunscreens; anesthetics; immunomodulators and nourishing agents; moisture absorbers; sebum absorbers, and mixtures thereof.

The polymer matrix of the present invention can also include as the active ingredient or as an additive one or more vegetable preparations, such as extracts or tinctures for the treatment of topical skin diseases. Suitable extracts or tinctures include oak bark extract, walnut extract, tincture of arnica, hamamelis extract, ribwort extract, pansy extract, thyme or sage extract; for the treatment of damaged or injured skin, for example, St. John's wort tincture, cone flowers tincture, chamomile flowers extract, or calendula flowers tincture; and for the care of exhausted and damaged skin, for example, birch leaf extract, nettle extract, coldsfoot extract, comfrey tincture, horsetail extract, or aloe vera extract. Vegetable preparations can also be released from the film layer for the intradermal treatment of diseases, for example, extracts of horse chestnut and butcher's broom in case of vein diseases, or extracts and tinctures of arnica, calendula, and capsicum in case of contusions, distortions, or hemorrhages. Vegetable preparations in the system according to the present invention may also be used in transdermal therapy, for example, ginseng extract in case of geriatric complaints; valerian tincture, extracts of melissa and hop to cause a sedative effect in case of superexcitation, sleep disturbances, and stress; extracts of kola and tea for stimulating; or hawthorn extract to stabilize the circulatory system.

Suitable amino acid agents that can be used in the present invention include amino acids derived from the hydrolysis of various proteins as well as the salts, esters, and acyl derivatives thereof. Non-limiting examples of such amino acid agents include amphoteric amino acids such as alkylamido alkylamines, stearyl acetyl glutamate, capryloyl silk amino acid, caprylol collagen amino acids; capryloyl kertain amino acids; capryloyl pea amino acids; cocodimonium hydroxypropyl silk amino acids; corn gluten amino acids; cysteine; glutamic acid; glycine; hair keratin amino acids; hair amino acids such as aspartic acid, threonine, serine, glutamic acid, proline, glycine, alanine, half-cystine, valine, methionine, isoleucine, leucine, tyrosine, phenylalanine, cysteic acid, lysine, histidine, arginine, cysteine, tryptophan, citrulline; lysine; silk amino acids, wheat amino acids; and mixtures thereof .

Suitable peptides, polypeptides, and proteins that can be used in the present invention include those polymers that have a long chain, such as at least about 10 carbon atoms, and a high molecular weight, such as at least about 1000, and are formed by self-condensation of amino acids. Examples of such proteins include collagen, deoxyribonuclease, iodized corn protein; keratin; milk protein; protease; serum protein; silk; sweet almond protein; wheat germ protein; wheat protein; wheat protein, alpha and beta helix of keratin proteins; hair proteins, such as intermediate filament proteins, high-sulfur proteins, ultrahigh-sulfur proteins, intermediate filament-associated proteins, high-tyrosine proteins, high-glycine tyrosine proteins, tricohyalin, and mixtures thereof.

Examples of suitable vitamins that can be used in the present invention include vitamin B complex; including thiamine, nicotinic acid, biotin, pantothenic acid, choline, riboflavin, vitamin B₆, vitamin B₁₂, pyridoxine, inositol, carnitine; vitamins A, C, D, E, K and their derivatives such as vitamin A palmitate and pro-vitamins, such as panthenol (pro vitamin B₅) and panthenol triacetate, and mixtures thereof.

Suitable antibacterial agents that can be used in the present invention include bacitracin, erythromycin, neomycin, tetracycline, chlortetracycline, benzethonium chloride, phenol, and mixtures thereof.

Examples of suitable skin emollients and skin moisturizers that can be used in the present invention include mineral oil, lanolin, vegetable oils, isostearyl isostearate, glyceryl laurate, methyl gluceth 10, methyl gluceth 20 chitosan, and mixtures thereof.

Examples of suitable hair conditioners that can be used in the present invention include quaternized compounds such as behenamidopropyl PG-dimonium chloride, tricetylammonium chloride, dihydrogenated tallowamidoethyl hydroxyethylmonium methosulfate, and mixtures thereof as well as lipophilic compounds like cetyl alcohol, stearyl alcohol, hydrogenated polydecene, and mixtures thereof.

Examples of sunscreen agents that can be used in the present invention include butyl methoxydibenzoylmethane, octyl methoxycinnamate, oxybenzone, octocrylene, octyl salicylate, phenylbenzimidazole sulfonic acid, ethyl hydroxypropyl aminobenzoate, menthyl anthranilate, aminobenzoic acid, cinoxate, diethanolamine methoxycinnamate, glyceryl aminobenzoate, titanium dioxide, zinc oxide, padimate, red petrolatum, -benzoyl-4-hydroxy-2-methoxy benzene sulfonic acid, 3,3′-(1,4 phenylenedimethylidene)-bis(7,7-dimethyl-2-oxo-bicyclo[2.2.1]heptane-1-methane sulfonic acid) sodium salt, mixtures of these compounds and others mentioned in Chapter 1 of “Sunscreens, Development, Evaluation and Regulatory Aspects,” edited by N. J. Lowe and N. A. Shaath, Marcel Dekker, Inc., 1990, which is incorporated herein by reference. An example of a suitable tanning agent is dihydroxyacetone. Examples of suitable skin lightening agents include hydroquinone, catechol and its derivatives, ascorbic acid and its derivatives, and mixtures thereof.

In the case of UV absorbers admixed with the polymers of the invention, it is often desirable to retain or fix the UV absorber within the dry film. One should then select a UV absorber very compatible with the polymer used, or select a polymer very compatible with the UV absorber used to achieve optimum retention of UV absorber in the film. A reason for retaining the UV absorber within the film is to protect skin tissue underneath the patch from ultraviolet light. The selection process may be done by one skilled in the art and through experimentation.

Examples of suitable depilating agents that can be used in the present invention include calcium thioglycolate, magnesium thioglycolate, potassium thioglycolate, strontium thioglycolate, and mixtures thereof.

Examples of suitable external analgesics and local anesthetics that can be used in present invention include benzocaine, dibucaine, benzyl alcohol, camphor, capsaicin, capsicum, capsicum oleoresin, juniper tar, menthol, methyl nicotinate, methyl salicylate, phenol, resorcinol, turpentine oil, and mixtures thereof.

Examples of suitable antiperspirants and deodorants that can be used in the present invention include aluminium chlorohydrates, aluminium zirconium chlorohydrates, and mixtures thereof.

Examples of suitable counterirritants that can be used in the present invention include camphor, menthol, methyl salicylate, peppermint and clove oils, ichtammol, and mixtures thereof.

An example of a suitable inflammation inhibitor that can be used in the present invention includes hydrocortisone.

Examples of suitable hemorrhoidal products that can be used in the present invention include anesthetics such as benzocaine, pramoxine hydrochloride, and mixtures thereof; antiseptics such as benzethonium chloride; astringents such as zinc oxide, bismuth subgallate, balsam Peru, and mixtures thereof; skin protectants such as cod liver oil, vegetable oil, and mixtures thereof.

Suitably, a type of active ingredient that can be used in the present invention includes those therapeutic agents that are effective in the treatment of seborrheic dermatitis, and psoriasis as well as the symptoms associated therewith. Examples of such suitable therapeutic agents include zinc pyrithione, shale oil and derivatives thereof such as sulfonated shale oil, selenium sulfide, sulfur; salicylic acid; coal tar; povidone-iodine and imidazoles.

Antimicrobials that can be used in the present invention for topical application are penicillins, cephalosporins, other beta-lactam compounds, aminoglycosides, tetracyclines, erythromycin, antifungal agents, and combinations thereof.

Antiseptics that can be used in the present invention for topical application onto acneiform skin are triclosan (Irgasan DP 300), phenoxy isopropanol, resorcinol, chlorhexidine, povidone and iodine.

Keratolytic agents that can be used in the present invention for topical application onto acneiform skin are salicylic acid, benzoyl peroxide, sulphur, retinoic acid and any of a number of fruit acids and alpha hydoxy acids.

Examples of anti-irritants that can be used in the present invention are alpha-bisabolol, farnesol, chamomile extract and glycyrrhetinic acid.

Examples of anti-cellulite agents that can be used in the present invention are caffeine and carnitine.

Examples of anti-inflammatory analgesic agents that can be used in the present invention include acetaminophen, methyl salicylate, monoglycol salicylate, aspirin, mefenamic acid, flufenamic acid, indomethacin, diclofenac, alclofenac, diclofenac sodium, ibuprofen, ketoprofen, naproxen, pranoprofen, fenoprofen, sulindac, fenclofenac, clidanac, flurbiprofen, fentiazac, bufexarnac, piroxicam, phenylbutazone, oxyphenbutazone, clofezone, pentazocine, mepirizole, and tiaramide hydrochloride. Examples of steroidal anti-inflammatory agents include hydrocortisone, predonisolone, dexamethasone, triamcinolone acetonide, fluocinolone acetonide, hydrocortisone acetate, predonisolone acetate, methylpredonisolone, dexamethasone acetate, betamethasone, betamethasone valerate, flumetasone, fluorometholone, and beclomethasone diproprionate.

Examples of antihistamines that can be used in the present invention include diphenhydramine hydrochloride, diphenhydramine salicylate, diphenhydramine, chlorpheniramine hydrochloride, chlorpheniramine maleate isothipendyl hydrochloride, tripelennamine hydrochloride, promethazine hydrochloride, methdilazine hydrochloride, and the like. Examples of local anesthetics include dibucaine hydrochloride, dibucaine, lidocaine hydrochloride, lidocaine, benzocaine, p-buthylaminobenzoic acid 2-(die-ethylamino)ethyl ester hydrochloride, procaine hydrochloride, tetracaine, tetracaine hydrochloride, chloroprocaine hydrochloride, oxyprocaine hydrochloride, mepivacaine, cocaine hydrochloride, piperocaine hydrochloride, dyclonine, and dyclonine hydrochloride.

Examples of bactericides and disinfectants that can be used in the present invention include thimerosal, phenol, thymol, benzalkonium chloride, benzethonium chloride, chlorhexidine, povidone iode, cetylpyridinium chloride, eugenol, and trimethylammonium bromide. Examples of vasoconstrictors include naphazoline nitrate, tetrahydrozoline hydrochloride, oxymetazoline hydrochloride, phenylephrine hydrochloride, tramazoline hydrochloride, and the like. Examples of hemostatics include thrombin, phytonadione, protamine sulfate, aminocaproic acid, tranexamic acid, carbazochrome, carbaxochrome sodium sulfanate, rutin, and hesperidin.

Examples of chemotherapeutic drugs that can be used in the present invention include sulfamine, sulfathiazole, sulfadiazine, homosulfamine, sulfisoxazole, sulfisomidine, sulfamethizole, and nitrofurazone. Examples of antibiotics that can be used in the present invention include penicillin, meticillin, oxacillin, cefalotin, cefalordin, erythromcycin, lincomycin, tetracycline, chlortetracycline, oxytetracycline, metacycline, chloramphenicol, kanamycin, streptomycin, gentamicin, bacitracin, and cycloserine.

Examples of antiviral drugs that can be used in the present invention include protease inhibitors, thymadine kinase inhibitors, sugar or glycoprotein synthesis inhibitors, structural protein synthesis inhibitors, attachment and adsorption inhibitors, and nucleoside analogues such as acyclovir, penciclovir, valacyclovir, and ganciclovir.

Examples of cosmetic active ingredients that can be used in the present invention are D-α-tocopherol, DL-α-tocopherol, D-α-tocopheryl acetate, DL-α-tocopheryl acetate, ascorbyl palmitate, vitamin F and vitamin F glycerides, vitamin D, vitamin D₂, vitamin D₃, retinol, retinol esters, retinyl palmitate, retinyl propionate, β-carotene, D-panthenol, famesol, farnesyl acetate; jojoba oils and blackcurrant oils rich in essential fatty acids; 5-n-octanoylsalicylic acid and esters thereof, salicylic acid and esters thereof; alkyl esters of α-hydroxy acids such as citric acid, lactic acid, glycolic acid; asiatic acid, madecassic acid, asiaticoside, total extract of centella asiatica, β-glycyrrhetinic acid, α-bisabolol, ceramides such as 2-oleoylamino-1,3-octadecane; phytanetriol, phospholipids of marine origin which are rich in polyunsaturated essential fatty acids, ethoxyquine; extract of rosemary, extract of balm, quercetin, extract of dried microalgae, anti-inflammatory agents, such as steroidal anti-inflammatory agents, and biostimulants, for example hormones or compounds for the synthesis of lipids and/or proteins.

Alpha-hydroxy acids (AHAs) can be used in the present invention as exfoliants, moisturizers, and emollients. Lactic acid salts, such as sodium lactate can be used in the present invention. In addition, AHAs and salicylic acid can be used in the present invention as a structurally similar β-hydroxy acid as peeling agents. The moisturizing activity of AHAs and their ability to exfoliate the skin and interfere with intercellular cohesion in the outer epidermis is well known. It has been suggested that AHAs interfere with cohesion in the stratum granulosum, unlike salicylic acid and other exfoliants.

Vitamin C (ascorbic acid) can be used in the present invention. Vitamin C promotes collagen (connective tissue) synthesis, lipid (fat) and carbohydrate metabolism, and the synthesis of neurotransmitters. Vitamin C is also essential for optimum maintenance of the body's immune system. Vitamin C is toxic to a wide range of cancer cells, especially melanoma. The oxidizing enzyme tyrosine that catalyzes the aerobic action of tyrosine into melanin and other pigments is also inhibited by the presence of vitamin C. Vitamin C has been found to be effective in catalyzing the immune response to many viral and bacterial infections. Besides the many applicable uses set forth above, vitamin C is essential for collagen synthesis and wound healing. The adhesive matrix of the present invention can comprise a combination of vitamin C, vitamin E and other ingredients, such as moisturizers, collagen synthesis promoting agents and exfoliating agents.

Skin treating compositions can be used in the present invention. Examples of skin treating compositions include vitamin C, vitamin C esters, vitamin E, vitamin E esters, and optionally, α-hydroxy acids, such as lactic and glycolic acids and other keratinolytics for the treatment or prevention of wrinkles and skin dryness.

Skin conditioners, moisturizers and surfactants can be included in the present invention. Illustrative conditioners include mineral oil, petrolatum, vegetable oils (such as soybean or maleated soybean oil), dimethicone, dimethicone copolyol, cationic monomers and polymers (such as guar hydroxypropyl trimonium chloride and distearyl dimethyl ammonium chloride) as well as combinations thereof. Illustrative moisturizers are polyols such as sorbitol, glycerin, propylene glycol, ethylene glycol, polyethylene glycol, polypropylene glycol, 1,3-butane diol, hexylene glycol, isoprene glycol, xylitol, fructose and mixtures thereof.

In another embodiment of the present invention, the active ingredient may be substantially fixed within the film layer upon so that the active ingredient is substantially non-migratory. In formulations where the active agent is non-migratory, desirably less than about 50 weight % of the active ingredient is transferred to the skin surface, such as, for example, less than about 25 weight %, or less than about 15 weight %, or even less than about 5 weight % in some embodiments. An example of such active agent is a light absorbing agent, such as an ultraviolet light absorber present in many sunscreens. These materials may be included in the polymer film by incorporating them into the polymer dispersions. These may include chemicals that absorb UVA and/or UVB radiation. These generally hydrophobic materials may be incorporated into the dispersion by a combination of heat, high shear or low shear stirring. These ingredients so incorporated may be organic or inorganic (such as titanium dioxide or zinc oxide, especially micro-fine grades with particle sizes of about 200 nanometers or less). When the polymer film dries on the skin, the UV absorbing chemicals may be held within the film and prevented from migrating into the skin, or possibly slowly released either into the skin or from the film into the environment depending on the design of the system. Suitable UV absorbers include those ingredients currently approved for use in the United States, Europe, and Japan. Non-limiting examples of such UV absorbers include butyl methoxydibenzoylmethane, octyl methoxycinnamate, oxybenzone, octocrylene, octyl salicylate, phenylbenzimidazole sulfonic acid, ethyl hydroxypropyl aminobenzoate, menthyl anthranilate, aminobenzoic acid, cinoxate, diethanolamine methoxycinnamate, glyceryl aminobenzoate, titanium dioxide, zinc oxide, padimate, red petrolatum, -benzoyl-4-hydroxy-2-methoxy benzene sulfonic acid, 3,3′-(1,4 phenylenedimethylidene)-bis[7,7-dimethyl-2-oxo-bicyclo(2.2.1)heptane-1-methane sulfonic acid] sodium salt, mixtures of these compounds.

A conventional surfactant or a combination of surfactants may be used as a stabilizer or solubility augmenting agent according to the present invention. For example, suitable surfactants generally include all pharmaceutically-acceptable surfactants, in which the surfactant has an HLB value of at least 10, and preferably at least about 15. HLB numbers and how they are determined for specific surfactants are well known to those skilled in the art. Generally, surfactants that can be used in the present invention are those selected from the anionic, cationic, nonionic, amphoteric, zwitterionic, and combinations thereof.

Examples of anionic surfactants include salts of sarcosinate, taurate, cocoyl isethionate, docusate salts such as the sodium salt thereof. In one embodiment, suitable pharmaceutically-acceptable anionic surfactants include, for example, those containing carboxylate, sulfonate, and sulfate ions such as for example, alkyl carboxylates, acyl lactylates, alkyl ether carboxylates, N-acyl sarcosinates, polyvalent alkyl carbonates, N-acyl glutamates, fatty acids having from 12 to 18 carbon atoms, polypeptide condensates and sulfuric acid esters. Other surfactants of choice include alkali or ammonium alkuylsulfate, alkylsulfonic acid or fatty acid, oxyethylated alkyl phenol, sulfosuccinates and their derivatives. A list of suitable surfactants is available in the treatise: McCutcheon's Emulsifiers and Detergents, North American Edition, MC Publishing Co, Glen Rock, N.J., 1997, the disclosure of which is incorporated herein by reference.

Those surfactants containing carboxylate ions are sometimes referred to as soaps and are generally prepared by saponification of natural fatty acid glycerides in alkaline solutions. Cations associated with these surfactants include sodium, potassium, ammonium and triethanolamine. The chain length of the fatty acids range from 12 to 18.

Examples of suitable amphoteric surfactants are cocoamidopropylbetaine, lauroamphoacetate, capryloamphopropionate, and disodium caprylohydroxypropyl sulfonate.

Examples of suitable non-ionics are trialkylamine oxides, alkyl polyglycosides and methyl glucamides. Suitable pharmaceutically-acceptable non-ionic surfactants include, for example, polyoxyethylene compounds, lecithin, ethoxylated alcohols, ethoxylated esters, ethoxylated amides, polyoxypropylene compounds, propoxylated alcohols, ethoxylated/propoxylated block polymers, propoxylated esters, alkanolamides, amine oxides, fatty acid esters of polyhydric alcohols, ethylene glycol esters, diethylene glycol esters, propylene glycol esters, glycerol esters, polyglycerol fatty acid esters, sorbitan esters, sucrose esters, glucose(dextrose)esters and simethicone.

Other surfactants that can be used in the present invention are sucrose distearate, diglyceryldistearate, tetraglyceryl tristearate, decaglyceryl decastearate, diglyceryl monostearate, hexaglyceyl tristearate, decaglyceryl pentastearate, sorbitan monostearate, sorbitan tristearate, diethylene glycol monostearate, the ester of glycerol and of palmitic acid and stearic acid, monostearate polyoxyethylenated containing 2 oxyethylene units, glyceryl mono- and dibehenate and pentaerythrityl tetrastearate

Examples of surfactants include, but are not limited to, alkali or ammonium alkysulfate, alkylsulfonic acid, or fatty acid having from 12 to 18 carbon atoms, oxyethylated alkyphenol, sulfosuccinates and derivatives, or any combination of anionic or non-ionic surfactants. Examples of suitable surfactant examples are lauryl sulfate, sodium salt, octylphenyl sulfonate, potassium salt, steric acid, ammonium salt, dodecyl sodium sulfosuccinate, and nonyl phenol 10 mole ethoxylate.

In addition to the above active ingredients a skin permeation enhancing agent or skin penetration enhancer may optionally be included in the polymer matrix. Agents known to accelerate the delivery of the drug through the skin have been referred to as skin-penetration enhancers, adjuvants, and absorption promoters, and are collectively referred to herein as “enhancers.” This class of agents includes those with diverse mechanisms of action including those which have the function of improving the solubility and diffusibility of the drug within the multiple polymers and those which improve percutaneous absorption, for example, by changing the ability of the stratum corneum to retain moisture, softening the skin, improving the skin's permeability, acting as penetration assistants or hair-follicle openers or changing the state of the skin including the boundary layer. Some of these agents have more than one mechanism of action, but in essence they serve to enhance the transdermal delivery of the drug. Some examples of enhancers are polyhydric alcohols such as dipropylene glycol, propylene glycol, and polyethylene glycol which enhance drug solubility; oils such as olive oil, squalene, and lanolin; fatty ethers such as acetyl ether and oleyl ether; fatty acid esters such as isopropyl myristate which enhance drug diffusibility; urea and urea derivatives such as allantoin which affect the ability of keratin to retain moisture; polar solvents such as dimethyldecylphosphoxide, methyloctylsulfoxide, dimethyllaurylamide, dodecylpyrrolidone, isosorbitol, dimethylacetonide, dimethylsulfoxide, decylmethylsulfoxide, and dimethylformamide which affect keratin permeability; salicylic acid which softens the keratin; amino acids which are penetration assistants; benzyl nicotinate which is a hair follicle opener; and higher molecular weight aliphatic surfactants such as lauryl sulfate salts which change the surface state of the skin; and the drugs to be administered. Other agents include oleic and linoleic acids, ascorbic acid, panthenol, butylated hydroxytoluene, tocopherol, tocopheryl acetate, tocopheryl linoleate, propyl oleate, and isopropyl palmitate.

Examples of suitable humectants for use in the layers of the composition and method of the invention are polyhydric alcohols such as glycerol, di-glycerol, triglycerol, polyglycerol, polyalkylene glycols and more preferably alkylene polyols and their derivatives, including propylene glycol, dipropylene glycol, polypropylene glycol, polyethylene glycol and derivatives thereof, sorbitol, hydroxypropyl sorbitol, hexylene glycol, 1,3-butylene glycol, isoprene glycol, 1,2,6-hexanetriol, ethoxylated glycerol, propoxylated glycerol and mixtures thereof. In particular, one found useful in small amounts is polyvinyl pyrrolidinone. Molecular weights suitable for this polymer when used to provide this functions are from about 1000 to about 100000 Daltons. This polymer may be used in combination with other humectants.

Plasticizers useful in this invention are generally diols, triols, polyols, alcohol ethers, alcohol esters, esters, ethers, hydroxy acids, amides, carbonates, and mixtures thereof. Suitable diols are 1,2-propylene glycol, ethylene glycol, 1,3 propylene glycol, 2-methyl-1,3-propanediol, butylene glycol, hexanediol, octanediol, and the like, containing up to 10 carbon atoms. Suitable triols are glycerin, trihydroxybutane, trihydroxyhexane, and the like. Alcohols having up to about six hydroxyl groups are suitable as plasticizers. Alcohol ethers suitable are diethylene glycol, dipropylene glycol, triethylene glycol, tetraethyene glycol, tripropylene glycol, and the like, alkoxylated alcohols such as ethoxylated alcohols, propoxylated alcohols, ethoxylated and propoxylated alcohols, where the alkoxylated alcohol is chosen from aliphatic, aromatic, alkaryl and aralkyl hydroxy functional compounds containing from 1 to 10 carbons and from one to six hydroxyl moieties. Examples of these are hydroquinone bis(hydroxyethyl ether), cyclohexanol hydroxyethyl ether, sorbitol trihydroxyethyl ether, catechol bis(hydroxyethyl ether), and mixtures thereof

Suitable alcohol ester plasticizers include propylene glycol acetate, glycerin diacetate (diacetin), ethylene glycol propionate, diethyl tartrate, diethyl citrate, triethyl citrate, tributyl citrate, sorbitol tetraacetate, propylene glycol mono-octoate, and the like. Suitable esters are triacetin, acetyl triethyl citrate, acetyl tributyl citrate, dimethyl malonate, dimethyl succinate, dimethyl adipate, diethyl malonate, diethyl oxylate, ethyl benzoate, and combinations thereof.

Suitable ether plasticizers include methoxybenzene, dimethoxy benzene, diethoxy benzene, triethylene glycol dimethoxyethylether, and the like. Suitable hydroxy acid plasticizers include glycolic, beta-hydroxy propionic acid, lactic acid, salicylic acid, citric acid, tartaric acid, and the like. Suitable amides include alkyl formamides such as methyl formamide, dimethyl formamide, diethyl formamide, hexyl formamide, acetamide, ethyl benzamide, N,N-diethyl acetamide, N-methyl pyrrolidinone, N-ethyl beta lactam, N-methyl caprolactam, caprolactam, N,N-dimethyldecanamide and the like. Suitable carbonates include ethylene carbonate, propylene carbonate, glycerol carbonate, sorbitol bis-carbonate, and the like. Care should be taken in selecting the materials suggested above for use as plasticizers for skin contact since some may have regulatory limitations when used on the skin.

Yet another class of plasticizers may have components of one or more of the classes noted above. Non-limiting examples include polyalkylene oxides, such as polyethylene glycol, polypropylene glycol, random or block polyethylene glycol-polypropylene glycol polymers, and random or block polyethylene glycol-polybutylene glycol. Other such polymer plasticizers include polyvinyl alcohol, polyhydroxyethyl cellulose, polyhydroxypropyl cellulose, hydroxyethyl guar, polyacrylamide, polyacrylic acid, polyacrylic acid-co-maleic acid, polyacrylamide-co-acrylic acid, carboxymethyl cellulose, carboxymethyl cellulose acetate-butyrate, poly(sodium vinyl benzene sulfonate) and copolymers and combinations thereof. Amounts of these polymeric plasticizers may be from about 1 to about 10 weight percent based on the weight of the water-dispersible or water dissipatable polymer of this invention.

Suitable agents may be added with the objective of controlling the polymer molecular weight. These are well known in the art, generally consisting of but not limited to mercaptans. Examples of these are alkyl mercaptans from 1 to about 20 carbons, mercapto acids and esters such as mercaptopropionic acid, alkyl mercaptopropioic acid esters, thiol glycols or dithioglycols such as hydroxyethyl mercapatan, thioglycerol,

The polymerization process of making modified “acrylic” latexes may also require adding to the monomer/hydrophobe mixture an initiator (oxidant), a reducing agent, or a catalyst. Suitable initiators include conventional initiators such as ammonium persulfate, sodium persulfate, hydrogen peroxide, t-butyl hydroperoxide, ammonium or alkali sulfate, di-benzoyl peroxide, lauryl peroxide, di-tertiarybutylperoxide, 2,2-azobisisobutyronitrile, benzoyl peroxide and mixtures thereof.

Suitable reducing agents are those which increase the rate of polymerization and include, for example, sodium bisulfite, sodium hydrosulfite, sodium formaldehyde sulfoxylate, ascorbic acid, isoascorbic acid, and mixtures thereof.

Suitable catalysts are those compounds which promote decomposition of the polymerization initiator under the polymerization reaction conditions thereby increasing the rate of polymerization. Suitable catalysts include transition metal compounds and driers. Examples of such catalysts include, but are not limited to, ferrous sulfate heptahydrate, ferrous chloride, cupric sulfate, cupric chloride, cobalt acetate, cobaltous sulfate, and mixtures thereof.

In another embodiment of the invention, a water-based latex containing acetoacetoxy ethylmethacrylate (referred to herein as AAEM) functionality acrylic, styrene/acrylic, or vinyl-acrylic resin (referred to herein simply as an ‘acrylic’ resin) containing petroleum jelly or Petrolatum and optionally an oily component such as coconut oil fatty acid. In the water-based latex, Petrolatum-coconut oil fatty acid modified acrylic resin generally exists as particles dispersed in water. The particles are generally spherical in shape. The particles may be structured (meaning non-homogeneous morphology) or unstructured. Structured particles include, but are not limited to core/shell particles and gradient particles. The core/shell polymer particles may also be prepared in a multi-lobe form, a peanut shell, an acorn form, or a raspberry form.

The miniemulsion polymerization process is used since miniemulsion allows the preparation of high molecular weight polymers at low viscosity unlike a solution or bulk polymerization. Shearing the mixture of monomer/water/surfactant/active agent forms small droplets of about 50 to 500 nanometers, and thus form a miniemulsion, prior to polymerization, ensuring that the polymerized latex therefrom contains the active agent dispersed uniformly throughout the latex particles. The Petrolatum or other active ingredient portion of the modified resin represents about 0.1 to about 50 weight %, such as, for example, from about 10 to about 40 weight % of total solids of the latex, or from about 10 to about 30 weight percent. If used in the miniemulsion process, the coconut oil fatty acid portion of the modified resin represents about 0.1 to about 20 weight % of the total solids of the latex. In another embodiment, the coconut oil fatty acid portion of the modified resin represents about 5 to about 15 weight % of the total solids of the latex.

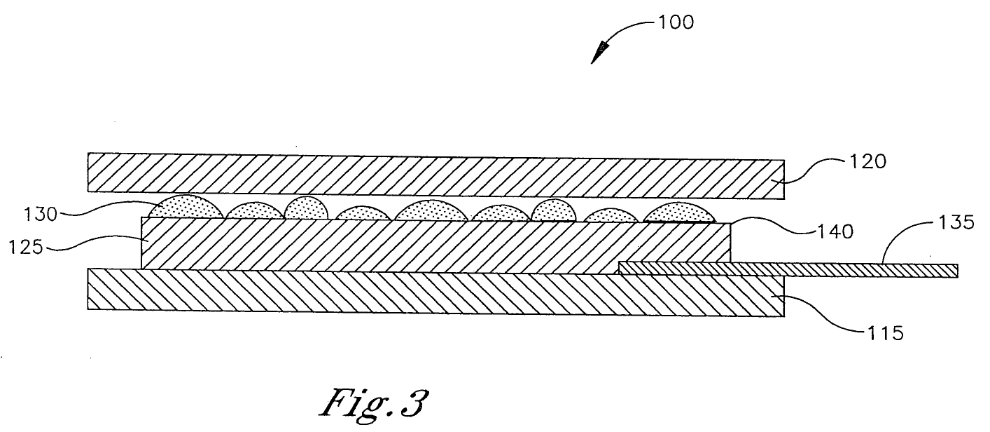

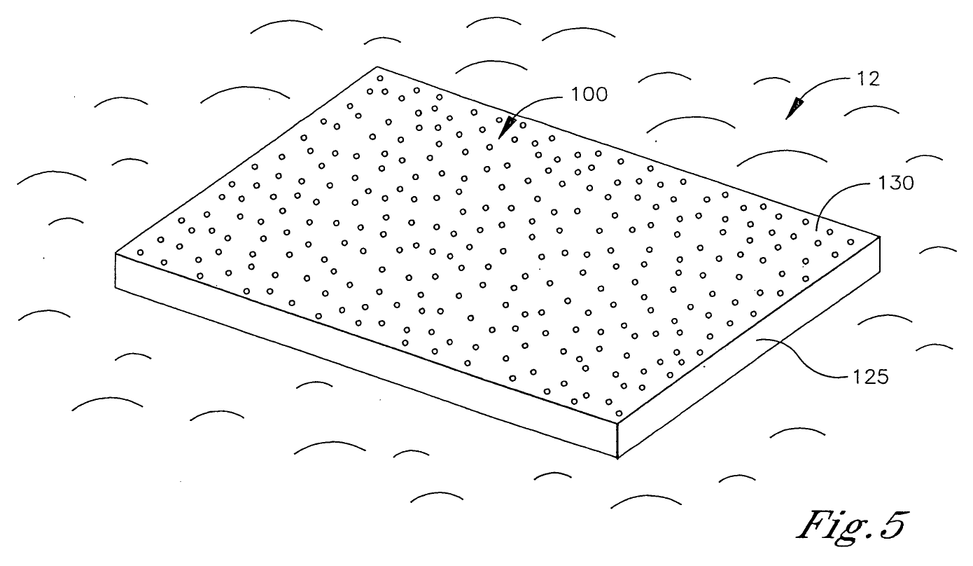

Referring to FIGS. 3 and 5, an alternative embodiment of the present invention is illustrated wherein at least one of the two layers is discontinuous. The patch 100 is similar to the patch 10 described above. That is, the patch 100 has two protective layers 115 and 120 covering the adhesive matrix first layer 125 and the second layer 130. Attached to one surface of the protective layer 115 is a means 135 for grasping the protective layer. However, the second layer 130 has a discontinuous surface area. As seen in FIG. 5, the discontinuous surface of the second layer substantially covers the entire upper surface 140 of the first layer. The discontinuous second layer 130 comprises a plurality of individual grains that substantially form a non-tacky surface. Advantageously, the grains individually have sufficient distance between an adjacent grain that the collective surface area is non-tacky, but at the same time have a sufficient porosity to allow moisture vapor transmission through the first layer 115. This allows the patch 100 to have a high degree of comfort and flexibility while allowing the tissue to remain cool and relatively dry.

Materials suitable for forming the discontinuous surface 130 can be any particulate material known to block or substantially reduce the tackiness of an adhesive. For example, the discontinuous layer 130 can be natural or synthetic crystalline or amorphous materials. Non-limiting examples of such materials include: starches such as corn, potato, rice, and wheat; mica; serecite; talcs; pigments; butters such as cocoa, Shea, kokum, mango, sal; clays such as bentonite, french green, fuller's earth, rhassoul, kaolin (white, pink, yellow, red and rose), green illite, blue montmorillonite, Moroccan red, multani mitti; waxes such as carnuba, beeswax, paraffin, synthetic waxes; rice bran; floral; hexagonal boron nitride ceramic powders; yucca shidigera powder; sodium ascorbyl phosphate; magnesium ascorbyl phosphate; hyaluronic acid; glass spheres, hollow glass spheres, ceramic spheres, silica spheres, alumina particles, polymer particles produced by grinding; poly(methyl methacrylate), polyethylene, ethylene/acrylate copolymer, Nylon-12, silicone resin, and polyurethane polymers of average diameter ranging from about 0.4 to about 14 micrometers; hollow spheres of ethylene/methacrylate copolymer of average diameters of from about 20 to about 32 micrometers; non-porous silica, porous silica, highly porous silica, and surface treated silica of average diameter of about 2 to about 12 micrometers; titanium dioxide, having particle sizes ranging from about 20 nanometers to about 300 nanometers; zinc oxide ranging in particle diameter from about 20 to about 500 nanometers or more, alumina powder, silica-alumina powder, polymers which are prepared by spray drying from water or organic solvents—powders having particle sizes of from about 0.5 to 30 micrometers, magnesium oxide powder and mixtures thereof.

In one embodiment, the discontinuous surface 130 is a polymeric material that has been applied to the upper surface 140 of the adhesive matrix layer 125. Such polymeric material should have a Tg of greater than about +5° C. and which may be “dusted” over the surface to reduce the upper surface tack.

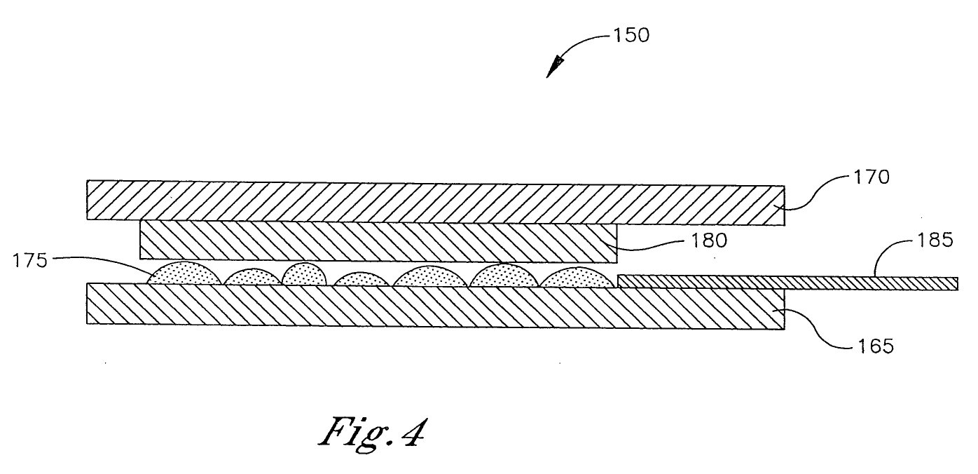

Referring to FIG. 4 an alternative embodiment of the present invention is illustrated, which is similar to that described above for FIG. 3, i.e., at least one of the two layers is discontinuous. The patch 150 is similar to patch 100 described above, that is, it has two protective layers 165 and 170, a first layer 175 and a second layer 180, and a grasping means 185, with the notable exception that in this embodiment the adhesive first layer 175 is discontinuous. This embodiment allows for use of a more aggressive or tacky adhesive since the adhesive is applied intermittently. The discontinuous first layer 175 comprises a plurality of individual grains that form an adhesive surface. Advantageously, this allows the manufacturer to more precisely control the adhesive force of the patch as well as allow site treatment using one or more active agents placed adjacent to the user's skin without substantial diffusion, interaction or interference between different types or concentrations of active agents.

Although the invention has been described as having the active agent included in the adhesive matrix of the first layer 25, 125, and 175, one skilled in the art would understand and fully appreciate that the active agent(s) may be included in the first layer 10, 125, and 175, or the second layer 30, 130 and 180, or both layers. Moreover, the active agent(s), their concentrations or composition may be the same or different in each layer. Thus, it is within the scope of the present invention for at least one layer to include a water-dispersible or water-dissipatable polymer and at least one layer include from about 0.1 to about 50 weight % of an active agent, and the two are not necessarily in the same layer. However, the sum of the weight percentages for each layer would still equal 100%. All such embodiments are within the teaching of the present invention.

Elongation: The elongation was measured on a free film (not in contact with a substrate or surface) according to the procedure of ASTM Method D882. The free film was prepared at a dry film thickness of 0.6 to 0.7 mil (0.0006 to 0.0007 inches thick) by making a drawdown with a suitable applicator on a release substrate. The release substrate may be any of a variety of substrates, so long as it is flat and non-porous, and have a low surface energy so that the film, when dry, may be readily separated from the substrate. Suitable substrates used include poly(tetrafluoroethylene), siliconized polyester film, siliconized paper, and wax paper. Drying conditions for this test were 22-25° C. air and substrate temperature for 24 hours. Afterwards the dried film was removed from the substrate and an elongation measured. In accordance with the present invention, the film must have an elongation of at least 50% and preferably is greater than 200%, when the sample was pulled at a rate of 10 inches per minute.

Another aspect of the present invention is a method for making the dermal patch of the present invention. The method comprises the steps of applying a first layer to a first protective releasable substrate; joining a second layer to the first layer; and covering the second layer with a second protective releasable substrate, wherein at least one of the first or second layers is a polymer matrix system having an active agent admixed therein, and at least one layer includes a water-dispersible or water-dissipatable polymer. Desirably, the first layer is dried before being joined with the second layer.

The present invention is illustrated in greater detail by the specific examples presented below. It is to be understood that these examples are illustrative embodiments and are not intended to be limiting of the invention, but rather are to be construed broadly within the scope and content of the appended claims. All parts and percentages in the examples are on a weight basis unless otherwise stated.

### EXAMPLE 1

Water-dispersible sulfopolyester A was prepared as follows: in a round bottom flask equipped with ground-glass head, an agitator shaft, nitrogen inlet and a side arm was charged with isophthalic acid, dimethyl-5-sodiosulfoisophthalate (SIP), diethylene glycol (DEG), and 1,4-cyclohexanedimethanol (CHDM), in the mole percents as set forth below. A catalyst was added and the flask was immersed in a Belmont bath at 200° C. for one hour under a nitrogen sweep. The temperature of the bath was increased to 230° C. for one hour. The temperature of the bath was increased to 280° C. and the flask was heated for 45 minutes under reduced pressure of 0.5 to 0.1 mm of Hg. The flask was allowed to cool to room temperature and the copolyester was removed from the flask and was extruded and pelletized.

Sulfopolyester A contained 18 mole percent dimethyl-5-sodiosulfoisophthalate and 82 mole percent isophthalic acid, and 46 mole percent 1,4-cyclohexanedimethanol and 54 mole percent diethylene glycol, based on 100 mole percent dicarboxylic acid and 100 mole percent diol. Sulfopolyester A has a Tg of 53° C. (as determined by differential scanning calorimetery) and an Inherent Viscosity (I.V.) of 0.33 dl/g was measured at 23° C. using 0.50 grams of polymer per 100 ml of a solvent consisting of 60% by weight phenol and 40% by weight tetrachloroethane).

A dispersion of the polymer pellets was prepared by heating to 80° C. 136 grams of deionized water in a 500 milliliter beaker. Then 64 grams of the polymer pellets were added with stirring, and the stirring continued for 30 minutes. The weight of the water that evaporated on heating was replaced as the formula cooled, giving a nearly clear polymer dispersion.

The following ingredients were placed in a 1 once wide-mouth jar:
1. 20.44 g of the sulfopolyester A polymer dispersion above;
2. 1.2 g Triacetin
3. 1.2 g DG Petroleum Jelly (available from Dolgen Corp., Inc., 100 Mission Ridge, Boodlettsville, Tenn. 37072); and
4. 0.47 g Clearate Lecithin (available from W.A. Cleary Corp., 1049 Route 27, P.O. Box 10, Somerset, N.J. 08875-0100.

The bottle was placed in a water-bath at 80° C. for 1 hour. The bottle was removed, and was shaken rapidly on a Brinkman Vibratory Mill until it was cool. The emulsion was creamy and stable to separation. The mixture was drawn down on a release film (Polyester Liner L-25X available from Sil-Tech, 222 Mound Avenue, Miamisburg, Ohio 45342) with a 0.004 of an inch gap film applicator. The coating was baked for 5 minutes at 90° C. Then an acrylic water based adhesive (Eastarez 2050 available from Hexion Specialty Chemicals, Columbus, Ohio 43215) was applied to the mixture coating using a number 0RK rod (approximately 0.15 mil wet) using a K-Control Coater (available from Testing Machine Company, 2 Fleetwood Court, Ronkonkoma, N.Y. 11779). The coating was dried for 5 minutes at 90° C. The film had an approximate final thickness of less than 1.0 mils (25 microns) for both layers. When applied to skin, the film transferred cleanly from the release polyester, and was not tacky or greasy to the touch. The film was washed off with soap and water after two hours. The skin beneath the film felt smooth to the touch.

### EXAMPLE 2

A miniemulsion having a Tg of 5° C. and 10 weight % Petrolatum and COFA was prepared as follows: To a 1000 mL resin kettle equipped with a condenser, nitrogen purge, and a subsurface feed tube was added 120 g of water. A nitrogen purge was begun and the contents heated and maintained at 80° C. Coconut Oil Fatty Acid (COFA), 41 grams, (C-108 obtained from Proctor and Gamble) was preheated at 60° C. and mixed with 41 grams of pre-heated (60° C.) petrolatum (purchased as Petroleum Jelly). The COFA-Petrolatum mixture in this example contained 10% Petrolatum (by weight of the total monomers).

This mixture was slowly added to the monomer mix and stirred for 3 hours to obtain a milky looking dispersion. The monomers mix consisted of 415.0 grams styrene/2-ethylhexyl acrylate/acetoacetoxy ethylmethacrylate/methacylic acid/acrylic acid. The weight ratio of monomers in the monomer mix was 44.5/43.2/9.4/0.7/2.2. Water (365 grams) and 18.3 grams of surfactant were premixed, then the monomer/Petrolatum/COFA mix was added to form a pre-emulsion. Surfactants included Aerosol OT-NV (available from Cytec Industries) and/or Hitenol BC1025 (available from DKS) in ratio of 1.1:0.4. The pre-emulsion was sheared using an IKA (Model SD-45) rotor/stator homogenizer by pumping through a flow cell which surrounded the shearing device with the homogenizer operating at maximum rpm to form a miniemulsion. Seventy-six grams (10%) of the miniemulsion was charged to the reactor. Then 0.6 g of ammonium persulfate was mixed in 10 g of water and charged to the reactor mixture and held at 80° C. After 15 minutes the remaining miniemulsion was fed over 180 minutes to the reactor. Simultaneously, an initiator feed composed of 79.0 g of water, 0.84 g of ammonium persulfate, and 0.84 g of ammonium carbonate was also fed over 180 minutes. After the feeds ended, the reactor was held at 80° C. for 60 minutes, before cooling to 50° C. Then a reductant solution consisting of 6.4 g water, 1.0 g isoascorbic acid, and 1.2 g of 0.5% iron sulfate heptahydrate, and 0.34 g of 28% ammonium hydroxide was added to the reactor. A solution of 19.0 g water and 1.10 g 70% t-butyl hydroperoxide was then fed over 48 minutes. The reaction mix was cooled to room temperature. The latex was filtered through a 100 mesh wire screen and filterable solids or scrap was determined as less than 0.1 weight %, based on the total batch weight. The droplet and particle sizes were measured using Microtrac UPA Particle Size Analyzer laser light-scaftering device (180 degree backscattering). For this particle size measurement the sample was diluted approximately 1:50 in water.

### EXAMPLE 3

Following the procedures of Example 2 above, a miniemulsion having a Tg of 5° C. and 20 weight % Petrolatum and COFA was prepared with the following exceptions, the petrolatum amount was 82 grams.

### EXAMPLE 4

A miniemulsion having a Tg of −30° C. and 20 weight % Petrolatum and COFA was prepared similar to that of Example 3 with the following exceptions, the styrene/2-ethylhexyl acrylate/acetoacetoxy ethylmethacrylate/methacylic acid/acrylic acid monomer ratios were 19.9/69.3/17.9/0.8/2.2.

### EXAMPLE 5

A miniemulsion having a Tg of −30° C. and 20 weight % Petrolatum was prepared similar to that of Example 4 with the following exceptions, the COFA was eliminated. This example shows that a miniemulsion of petrolatum and monomers can be prepared using a high shear condition which could not otherwise be achieved using a conventional emulsion processes.

### EXAMPLE 6

A miniemulsion having a Tg of −30° C. and 20 weight % Shea Butter was prepared similar to that of Example 5 with the following exceptions, the petrolatum was replaced by Shea Butter.

### EXAMPLE 7

Example 7 was prepared similar to that of Example 5 except that the monomer mix ratio was as follows: 2-ethylhexyl acrylate/methacrylic acid 65.0/35.0.

### EXAMPLE 8

The liquid mixture from Example 1 was drawn down on release film (Polyester Liner L-25X, same film used in Example 2) with a 4 mil gap thin film applicator. The coating was baked for 5 minutes at 90° C. Then the miniemulsion from Example 4 was applied to the mixture coating above using a number 4RK wire wound rod (approximately 1.5 mil wet) and a K-Control Coater. This newly applied coating on the above film was dried for 5 minutes at 90° C. The resulting two-layer, dry film of about 1.5 mil thickness, when applied to skin, transferred to the skin cleanly from the release polyester, and was not tacky or greasy to the touch. The film was washed off with soap and water after two hours. The skin beneath the film felt smooth to the touch.

### EXAMPLE 9

A 25% solution of carboxymethylcellulose acetate butyrate (CMCAB) in (90%/10% isopropyl alcohol and water) was drawn down on release film (Polyester Liner L-25X) using a number 0RK rod and a K-Control Coater. The film was dried 30 seconds at 100° C. The mini-emulsion from Example 4 was applied to the top of the previously applied coating with a number 6 RK Rod (2.5 mil wet film). The coating was dried for 5 minutes at 100° C. The resulting dry film of about less than 1 mil thickness, when applied to skin, transferred to the skin cleanly from the release polyester, and was not tacky or greasy to the touch. The film was peeled off after 30 minutes. The skin beneath the film felt smooth to the touch.

This example illustrates that no pressure device is needed to force together the adhesive and the active ingredient, since the active ingredient is contained within the miniemulsion polymer already when it is applied. Applying pressure to adhesives require not only excess energy, but also something to press onto the adhesive, which tends to stick to the adhesive and become dirty, or it requires an additional layer (another release liner for the adhesive side) to protect the adhesive layer from sticking to the press.

### EXAMPLE 10

This example shows a film composition wherein both layers are prepared from two different miniemulsion compositions where each miniemulsion polymer has a Tg different than the other. The lower Tg polymer (−30° C.) resides on the side to be applied to the skin, and the second miniemulsion has a Tg of 5° C.

The latex from Example 3 (11.87 g) was combined with 3.52 g of a 10% solution Vinol 523 (polyvinyl alcohol thickener available from Air Products, Inc.) and mixed by shaking. The blend was drawn down using a 5 mil gap applicator on Polyester Liner L-25X available from Sil-Tech, and the resultant film was baked at 100° C. for 5 minutes. The film had a thickness of 0.016 millimeter and an elongation (determined by ASTM method D882) of 676 percent before breaking.

A similar film to that above was further coated using a 0RK wire wound rod with the polymer emulsion of Example 4. The film composite was baked for 5 minutes at 100° C. When cool, the film was readily transferred to the skin on the back of the hand. The polyester liner backing was readily removed by peeling, leaving the miniemulsion composite film on the skin. The film was water resistant. The film remained on the skin for about 2 hours. Upon removal by peeling from the skin, the skin felt smooth to the touch.

### EXAMPLE 11

A dispersion similar to Example 1 (modified to include 20% petrolatum) was drawn down Polyester Liner L-25X available from Sil-Tech with a 5 mil gap applicator, and the resultant film was baked at 100° C. for 5 minutes. A similar film to that above was further coated using a 0RK wire wound rod with the polymer emulsion of Example 6. The film composite was baked for 5 minutes at 100° C. When cool, the film was readily transferred to the skin on the back of the hand. The polyester liner backing was readily removed by peeling, leaving the miniemulsion composite film on the skin. The film remained on the skin for about 2 hours. Upon removal of the film by washing with soap and water, the skin felt smooth to the touch.

### EXAMPLE 12

The following ingredients were placed in a 4 once wide-mouth jar added in the order listed while mixing with a Dispax high speed disperser equipped with a fine head available from IKA® WORKS, INC., Wilmington, N.C. The mixer speed was controlled using a POWERSTAT® Variable Transformer available from Superior Electric, Bristol, Conn. Initial mixing was done at 40 volts (slow speed).
1. 131.49 g of the polymer dispersion of Example 1;
2. 7.72 g Triacetin;
3. 10.5 g Avocado Oil (Jan Dekker International, Plein 13 no. 1, 1521 AP Wormerveer, Netherlands); and
4. 4.1 g Thermalec 57.

The mixing speed was increased by applying 60 volts to the transformer. The temperature of the mixture was 63° C. The emulsion was smooth and creamy. The jar was capped, and it was placed in a water bath at 85° C. for 1 hour. The mixture was then dispersed at 60 volts for 3 minutes, then stirred using a motorized slow speed agitator until it reached room temperature. The emulsion was thin and creamy.

The mixture was drawn down on a release film (Polyester Liner L-25X) with a 4 mil gap film applicator. The coating was baked for 5 minutes at 90° C. Then an acrylic water based adhesive (Eastarez 2050 available from Hexion Specialty Chemicals, Columbus, Ohio 43215.) was applied to the mixture coating using a number 0RK rod (approximately 0.15 mil (4 micrometer) wet) using a K-Control Coater (available from Testing Machine Company, 2 Fleetwood Court, Ronkonkoma, N.Y. 11779). The coating was dried for 5 minutes at 90° C. The film had an approximate final thickness of less than 1.0 mil for both layers. When applied to skin, the film transferred cleanly from the release polyester and was not tacky or greasy to the touch. The film was washed off with soap and water after two hours. The skin which had been beneath the film felt smooth to the touch.

### EXAMPLE 13

A miniemulsion having a Tg of 5° C. and 10 weight % COFA was prepared as follows.

To a 1000 mL resin kettle equipped with a condenser, nitrogen purge, and a subsurface feed tube was added 119.3 g of water. A nitrogen purge was begun and the contents heated and maintained at 80° C. Coconut Oil Fatty Acid (COFA), 44.0 grams, (C-108 obtained from Proctor and Gamble) was preheated at 60° C. and slowly added to a monomer mix consisting of 187.7 grams styrene, 182.6 g 2-ethylhexyl acrylate, 39.8 g acetoacetoxy ethylmethacrylate, 3.11 g methacylic acid, and 9.32 g acrylic acid. The mixture was stirred for 3 hours to obtain a milky looking dispersion.

A surfactant mixture was prepared by adding 385.9 grams of water, 5.51 g Aerosol OT-NV (available from Cytec Industries) and 7.41 g Hitenol BC1025 (available from DKS). Then the monomer-COFA mixture above was added to form a pre-emulsion. The pre-emulsion was sheared using an IKA (Model SD-45) rotor/stator homogenizer by pumping through a flow cell which surrounded the shearing device with the homogenizer operating at maximum rpm to form a miniemulsion. Ten weight percent (72.2 grams) of the miniemulsion was charged to a reactor held at 80° C. Then 0.58 g of ammonium persulfate was mixed with 9.75 g of water and charged to the reactor mixture, still held at 80° C. After 15 minutes the remaining miniemulsion was fed over 180 minutes to the reactor. Simultaneously, an initiator feed composed of 78.0 g of water, 0.83 g of ammonium persulfate, and 0.83 g of ammonium carbonate was also fed over 180 minutes. After the feeds ended, the reactor was held at 80° C. for 60 minutes, before cooling to 50° C. Then a reductant solution consisting of 6.34 g water, 1.0 g isoascorbic acid, and 1.2 g of 0.5% iron sulfate heptahydrate, and 0.34 g of 28% ammonium hydroxide was added to the reactor. A solution of 19.0 g water and 1.10 g 70% t-butyl hydroperoxide was then fed over 48 minutes. The reaction mix was cooled to room temperature. The latex was filtered through a 100 mesh wire screen and filterable solids or scrap was determined as less than 0.1% based on the total batch weight. The particle size was 390 nanomters as measured by using a Microtrac UPA Particle Size Analyzer—laser light-scattering device (180 degree backscattering). For this particle size measurement the sample was diluted approximately 1:50 in water.

### EXAMPLE 14

Sulfopolyester B was Prepared as Follows.

In equipment similar to that used in Example 1, the following composition was heated in a similar fashion: 11 mole percent dimethyl-5-sodiosulfoisophthalate and 89 mole percent isophthalic acid, and 21.5 mole percent 1,4-cyclohexanedimethanol and 78.5 mole percent diethylene glycol, based on 100 mole percent dicarboxylic acid and 100 mole percent diol. The resultant Sulfopolyester B has a Tg of 35° C. and an I.V. of 0.32 dl/g.

A dispersion of the polymer pellets was prepared by heating to 80° C., 136 grams of deionized water in a 500 milliliter beaker. Then 64 grams of the polymer pellets were added with stirring, and the stirring continued for 30 minutes. The weight of the water that evaporated on heating was replaced as the formula cooled, giving a slightly turbid polymer dispersion.

### EXAMPLE 15

This example illustrates the use of two different active ingredients in different polymer layers. Part A was prepared as follows. The following ingredients were placed in a 1 ounce wide-mouth jar:
1. 14.97 g of the Mini-emulsion from Example 14;
2. 3.0 g Vinol 540, (polyvinyl alcohol thickener available from Air Products, Inc.)10% solution;
3. 1.5 g n-propyl alcohol;
4. 2.13 g Colavita Extra Virgin Olive Oil (available from Colavita USA, Linden, N.J.); and
5. 1.1 g EASTMAN EB (hydroxyethyl butyl ether).

The bottle was shaken without heating for 15 minutes. The emulsion was creamy and stable to separation. The mixture was drawn down on a release film (Polyester Liner L-25X available from Sil-Tech, Miamisburg, Ohio.) with a #8RK rod. The coating was baked for 5 minutes at 100° C. The resulting film was smooth, tacky and translucent.

Part B was prepared as follows. The following ingredients were placed in a 1 ounce wide-mouth jar:
1. 3.37 g Vinol 540, (polyvinyl alcohol thickener available from Air Products, Inc.) 10% solution;
2. 20.44 g Sulfopolyester Dispersion from Example 14;
3. 1.6 g NutriLayer Phytolipid (available from Eastman Chemical Company);
4. 0.68 g Coconut Oil Fatty Acid; and
5. 0.96 g Triacetin.

The sample was heated at 85° C. for 30 minutes. The sample was then shaken on a Brinkman Vibratory Mill until it was cool. The emulsion was thin and creamy. Additional solvent, 1.1 g of EASTMAN EB was added to help wet the release film (Polyester Liner L-25X available from Sil-Tech, Miamisburg, Ohio). The emulsion was applied with a #8RK rod. The coating was baked for 5 minutes at 100° C. The film was smooth, not tacky and translucent.

Part A was applied to a coating described in Part B. Both layers were applied with a #8 RK Rod. The two-layer coating was baked at 100° Centigrade for 5 minutes. The coating was easily transferred from the release film onto the skin. The coating was removed by peeling.

With a #0 RK rod, the polymer from Example 6 was applied to a coating described in Part B. The two-layer coating was baked at 100° C. for 5 minutes. The resulting coating was only slightly tacky, but it was easily transferred from the release film onto the skin by applying hand pressure for less than 30 seconds. The coating was removed by washing with soap and water or by peeling.

### EXAMPLE 16

A miniemulsion having a Tg of −5° C. and a high concentration of Petrolatum (20 weight %) was prepared as follows:

Water (120 grams ) were added to a 1000 mL resin reactor equipped with a condenser, nitrogen purge, and a subsurface feed tube. A nitrogen purge was initiated and the contents were heated to 80° C.

A monomers premix was prepared having:
1. 345.0 grams of 2-ethylhexyl acrylate/methacrylic acid in the weight ratio of 65/35,
2. 300 grams of water, and
3. 15.5 grams of a surfactant blend having Aerosol OT-NV (available from Cytec Industries) and/or Hitenol BC1025 (available from DKS) in ratio of 1.1:0.4.

Petrolatum (69 grams), purchased as Petroleum Jelly, was slowly added to the monomers premix and stirred for 3 hours to obtain a milky looking dispersion. The dispersion was sheared using an IKA (Model SD-45) rotor/stator homogenizer by pumping the dispersion through a flow cell operating at maximum rpm to form a miniemulsion.

A reaction initiator feed was prepared having 90.0 g of water, 1 g of ammonium persulfate, and 1 g of ammonium carbonate.

Seventy-two grams (about 10%) of the miniemulsion was charged to the reactor.

Ammonium persulfate (0.65 g) was mixed in 12 g of water and charged to the reactor mixture. After 15 minutes, the remaining miniemulsion was fed to the reactor over a period of 180 minutes. Concurrently with the miniemulsion feed, the initiator feed was also fed to the reactor but over a time period of 195 minutes.

After the feeds ended, the reactor was held at 80° C. for 60 minutes, then cooled to 50° C. A reductant solution consisting of 10 g water, 1.0 g isoascorbic acid, and 1.2 g of 0.5% iron sulfate heptahydrate, and 0.34 g of 28 weight % ammonium hydroxide was added to the reactor.

A solution of 25.0 g water and 1.2 g 70 weight % t-butyl hydroperoxide was then fed to the reactor over a period of 48 minutes. The reaction product was then cooled to room temperature. The resulting latex was filtered through a 100 mesh wire screen and filterable solids (scrap) was determined as less than 0.1 weight %, based on the total batch weight. Mean particle size of the finished latex was 328 nm. The droplet and latex particle sizes were measured using Microtrac UPA Particle Size Analyzer laser light-scattering device (180° backscattering). To determine particle size, the sample was diluted in water at a ratio of approximately 1:50 v/v. The resulting miniemulsion latex was drawn down to form a film on glass, then heated for 5 minutes at 80° C. to drive off the water. The film was found to be readily removable from the glass surface with mild rubbing with water.

### EXAMPLE 17

This example illustrates a composition wherein both layers are composed of compositions removable from the skin by water washing.

Aqueous Phase: To a beaker were added sequentially with mild stirring while heating to 75° C. the following ingredients:
1. 71.4 g of a 32% solids dispersion of Eastman AQ55 polyester;
2. 11.3 g deionized water;
3. 1.83 g glycerin;
4. 0.57 g of polyvinylpyrrolidinone polymer of 90,000 molecular weight;
5. 0.05% disodium EDTA dehydrate; and
6. 0.76 grams cetyl phosphate, potassium salt (Amphisol K, available from DSM).

Organic Phase: In a separate beaker were added the organic phase ingredients, also with stirring and heating to 80 to 90° C. the following components:
1. 5.00 g Petrolatum;
2. 2.29 grams triethyl citrate;
3. 1.52 g cetearyl alcohol (Lanefte O available from Cognis); and
4. 1.00 g Glycerol monostearate (Cutina GMS V, available from Cognis).

The hot organic phase was then poured into the warm aqueous phase with stirring. Then 4.00 g of Silica MSS-500/3H4 was added with stirring. Then the entire mixture was homogenized using a rotor stator mixer for 10 minutes at 8000 rpm. The resulting emulsified blend was stirred with low shear rate stirring until cool. Then a preservative, 0.48 grams of Phenonip (available from Clariant International, Ltd.) was stirred into the blend.

A drawdown of the mixture above was prepared, using a wire wound drawbar, on siliconized polyester substrate (Polyester Liner L-25X available from Sil-Tech, 222 Mound Avenue, Miamisburg, Ohio 45342) having a thickness of about 1 mil. The dried film, measured after about 1 hour of drying at ambient conditions had a film thickness of about 1 mil.

A second drawdown was made exactly on top of the dried film above, using a water dispersible polyester adhesive blend and the same draw bar used above. This adhesive blend was prepared previously by heating together the following ingredients: i) 10.69 g AQ55 polyester (34.6% solids in water); ii) 60.04 g AQ1045 polyester (30.8% solids in water); iii) 0.92 g glycerin; and iv) 0.99 g triethyl citrate.

After vigorous shaking the mixture in a vial to mix, the contents were warmed to about 80° C. in a microwave oven, then re-shaken again to insure homogeneity. The blend then was allowed to rest undisturbed for two weeks, during which time it developed its full adhesive power when converted to a dry film. A dry film of this blend had a Tg −4° C., and was very tacky to the touch.

After drying the top adhesive layer overnight at room temperature, a patch was constructed by applying Scotch Magic tape to one edge of the dried film, overlapping the tape onto the siliconized polyester substrate, then covering with a top sheet of the same siliconized polyester used for the initial coating substrate. Rectangular patches were cut from the sheet, with the tape along one edge only.

The dry patch was applied to the skin of the volar wrist of a male volunteer. After two hours of wearing the patch, the patch was removed by washing with warm tap water and gentle rubbing. A day later, a second patch applied on the wrist was worn for 8 hours, and was removable in the same way. A day later a third patch worn on the wrist for 4 hours was removed using warm soapy water with gentle rubbing. No cracking of any of the films was observed while on the skin. No residue was apparent after any of the washings.

Having described the invention in detail, those skilled in the art will appreciate that modifications may be made to the various aspects of the invention without departing from the scope and spirit of the invention disclosed and described herein. It is, therefore, not intended that the scope of the invention be limited to the specific embodiments illustrated and described but rather it is intended that the scope of the present invention be determined by the appended claims and their equivalents. Moreover, all patents, patent applications, publications, and literature references presented herein are incorporated by reference in their entirety for any disclosure pertinent to the practice of this invention.

What is claimed is:

1. A dermal patch comprising a first and a second layer, wherein at least one of said layers is a polymer matrix system having an active agent admixed therein, wherein at least one layer comprises a water-dispersible or water-dissipatable film-forming polymer, and wherein the dermal patch has an elongation factor of at least 50%.

2. The dermal patch of claim 1 wherein said water-dispersible or water-dissipatable film-forming polymer is selected from the group consisting of sulfopolyesters, sulfonated acrylics, hybrid polymers, acrylic polymers or mixtures thereof.

3. The dermal patch of claim 2 wherein said water-dispersible or water-dissipatable acrylic polymer is prepared using a miniemulsion process to form a latex.

4. The dermal patch of claim 2 wherein the first layer is an adhesive matrix and the second layer is substantially non-tacky.

5. The dermal patch of claim 4 wherein the first layer has a Tg of from about −5° C. to about −45° C. and the second layer has a Tg greater than about 5° C., and wherein the difference between the first layer Tg and the second layer Tg is at least about 15° C.

6. The dermal patch of claim 4 wherein the difference between the first layer Tg and the second layer Tg is at least about 25° C.

7. The dermal patch of claim 2 wherein the first layer comprises: a) from about 25 to about 99.8 weight % of the film-forming polymer, and b) from about 0.1 to about 50 weight % of an active agent, wherein the sum of the ingredients equals 100 weight %.

8. The dermal patch of claim 2 wherein the first layer comprises: a) from about 30 to about 95 weight % of the film-forming polymer; and b) from about 1 to about 40 weight % of an active agent, wherein the sum of the ingredients equals 100 weight %.

9. The dermal patch of claim 2 wherein the first layer comprises: a) from about 40 to about 95 weight % of the film-forming polymer; and b) from about 1 to about 40 weight % of an active agent, wherein the sum of the ingredients equals 100 weight %.

10. The dermal patch of claim 7, 8 or 9 further comprising at least one of: c) from about 0.1-25 weight % of a surfactant; or d) less than about 10 weight % of a skin permeation enhancing agent; or e) up to about 20 weight % of a humectant; or f) up to about 20 weight % of a plasticizer, wherein the sum of the ingredients equals 100 weight %.

11. The dermal patch of claim 2 wherein said first layer comprises a sulfopolyester.

12. The dermal patch of claim 2 wherein said first layer comprises an acrylic polymer.

13. The dermal patch of claim 1 wherein said active ingredient is selected from the group consisting of anti-oxidants; free radical scavengers; skin moisturizers; depigmentation agents; reflectants; humectants; antimicrobial (e.g., antibacterial) agents; allergy inhibitors; anti-acne agents; anti-aging agents; anti-wrinkling agents, antiseptics; analgesics; antitussives; antipruritics; local anesthetics; antihistamines; keratolytic agents; anti-inflammatory agents; fresheners; healing agents; anti-infectives; inflammation inhibitors; anticholinergics; vasoconstrictors; vasodilators; wound healing promoters; peptides, polypeptides and proteins; deodorants and antiperspirants; skin emollients; tanning agents; skin lightening agents; antifungals such as antifungals for foot preparations; depilating agents; external analgesics; counterirritants; insecticides; poison ivy products; poison oak products; burn products; anti-diaper rash agents; prickly heat agents; make-up preparations; vitamins; amino acids and their derivatives; herbal extracts; retinoids; flavoids; sensory markers (i.e., cooling agents, heating agents, etc.); skin conditioners; chelating agents; cell turnover enhancers; coloring agents; sunscreens; anesthetics; immunomodulators and nourishing agents; moisture absorbers; sebum absorbers, or mixtures thereof.

14. The dermal patch of claim 12 wherein said acrylic polymer is a copolymer further comprising up to about 40 mole % of a co-monomer selected from the group consisting of styrene, alpha-methyl styrene, vinyl naphthalene, vinyl toluene, and chloromethyl styrene, methyl acrylate, acrylic acid, methacrylic acid, methyl methacrylate, ethyl acrylate, ethyl methacrylate, butyl acrylate, butyl methacrylate, isobutyl acrylate, isobutyl methacrylate, ethylhexyl acrylate, ethylhexyl methacrylate, octyl acrylate, octyl methacrylate, glycidyl methacrylate, carbodiimide methacrylate, alkyl crotonates, vinyl acetate, di-n-butyl maleate, and di-octylmaleate, t-butylaminoethyl methacrylate, dimethylaminoethyl methacrylate, diethylaminoethyl methacrylate, N,N-dimethylaminopropyl methacrylamide, 2-t-butylaminoethyl methacrylate, N,N-dimethylaminoethyl acrylate, N-(2 methacryloyoxy-ethyl)ethylene urea or mixtures thereof.

15. The dermal patch of claim 10 wherein said surfactant is selected from anionic or non-ionic surfactants.

16. The dermal patch of claim 15 wherein said surfactant is an anionic surfactant selected from the group consisting of alkyl carboxylates, acyl lactylates, alkyl ether carboxylates, N-acyl sarcosinates, polyvalent alkyl carbonates, N-acyl glutamates, fatty acid, polypeptide condensates, or sulfuric acid esters.

17. The dermal patch of claim 13 wherein said active ingredient is petrolatum.

18. The dermal patch of claim 3 wherein said acrylic latex has an average particle size of from about 25 to about 500 nm.

19. The dermal patch of claim 18 wherein said acrylic latex has an average particle size of from about 50 to about 300 nm.

20. The dermal patch of claim 10 wherein said surfactant is selected from the group consisting of sarcosinate, taurate, cocoyl isethionate, alkali metal docusate salts, alkyl carboxylates, acyl lactylates, alkyl ether carboxylates, N-acyl sarcosinates, polyvalent alkyl carbonates, N-acyl glutamates, fatty acids having from 12 to 18 carbon atoms, polypeptide condensates, sulfuric acid esters, dialkylamine oxides, alkyl polyglycosides and methyl glucamides, polyoxyethylene compounds, lecithin, ethoxylated alcohols, ethoxylated esters, ethoxylated amides, polyoxypropylene compounds, propoxylated alcohols, ethoxylated/propoxylated block polymers, propoxylated esters, alkanolamides, amine oxides, fatty acid esters of polyhydric alcohols, ethylene glycol esters, diethylene glycol esters, propylene glycol esters, glycerol esters, polyglycerol fatty acid esters, sorbitan esters, sucrose esters, glucose esters, or simethicone.

21. The dermal patch of claim 1 wherein said active agent is fugitive and from about 1 to about 100% of the fugitive active agent transfers from the dermal patch to the epidermis of the user.

22. The dermal patch of claim 1 wherein said active agent is fugitive and from about 10 to about 100% of the fugitive active agent transfers from the dermal patch to the epidermis of the user.

23. The dermal patch of claim 1 wherein said active agent is substantially fixed in at least one layer and wherein less than about 25% of the active agent is transferred to the epidermis of the user.

24. The dermal patch of claim 1 wherein said active agent is substantially fixed in at least one layer and wherein less than about 15% of the active agent is transferred to the epidermis of the user.

25. The dermal patch of claim 1 wherein said active agent is substantially fixed in at least one layer and wherein less than about 5% of the active agent is transferred to the epidermis of the user.

26. The dermal patch of claim 23, 24, or 25 wherein said active agent is a UV absorbing compound selected from the group consisting of butyl methoxydibenzoylmethane, octyl methoxycinnamate, oxybenzone, octocrylene, octyl salicylate, phenylbenzimidazole sulfonic acid, ethyl hydroxypropyl aminobenzoate, menthyl anthranilate, aminobenzoic acid, cinoxate, diethanolamine methoxycinnamate, glyceryl aminobenzoate, titanium dioxide, zinc oxide, padimate, red petrolatum, -benzoyl-4-hydroxy-2-methoxy benzene sulfonic acid, 3,3′-(1,4 phenylenedimethylidene)-bis[7,7-dimethyl-2-oxo-bicyclo(2.2.1) heptane-1-methane sulfonic acid]sodium salt, or mixtures of these compounds.

27. The dermal patch of claim 10 wherein said plasticizer is selected from the group consisting of diols, triols, polyols, alcohol ethers, alcohol esters, esters, ethers, hydroxy acids, amides, carbonates, or mixtures thereof.

28. The dermal patch of claim 10 wherein said plasticizer is selected from the group consisting of triacetin, triethyl citrate, glycerin, sorbitol, 1,2-propylene glycol, ethylene glycol, 1,3 propylene glycol, 2-methyl-1,3-propanediol, butylene glycol, hexylene glycol, isoprene glycol, xylitol, fructose, hexanediol, octanediol, or mixtures thereof.

29. The patch of claim 1 wherein said patch has an elongation of at least 200%.

30. A dermal patch comprising a first layer and a second layer, wherein at least one layer comprises from about 25 to about 99.8 weight % of a water-dispersible or water-dissipatable film-forming polymer and at least one layer comprises from about 0.1 to about 50 weight % of an active agent, wherein the sum of the ingredients in each layer equals 100 weight %.

31. The dermal patch of claim 30 wherein said water-dispersible or water-dissipatable film-forming polymer is selected from the group consisting of sulfopolyesters or acrylic polymers.

32. The dermal patch of claim 30 wherein at least one layer further comprises one or more of: a) from about 0.1 to about 25 weight % of a surfactant; or b) less than about 10 weight % of a skin permeation enhancing agent; or c) up to about 20 weight % of a humectant; or d) up to about 20 weight % of a plasticizer, wherein the sum of the ingredients equals 100 weight %.

33. The dermal patch of claim 30 wherein at least one layer is discontinuous.

34. The dermal patch of claim 30 wherein said first layer is an adhesive layer and said adhesive layer is discontinuous.

35. The dermal patch of claim 30 wherein said second layer is a non-tacky layer and said non-tacky layer is discontinuous.

36. The dermal patch of claim 35 wherein said discontinuous second layer comprises particulate materials known to block or substantially reduce the tackiness of an adhesive.

37. The dermal patch of claim 36 wherein said discontinuous second layer is selected from the group consisting of corn, potato, rice, and wheat starches; mica; serecite; talcs; pigments; cocoa, Shea, kokum, mango, and sal butters; clays selected from bentonite, french green, fuller's earth, rhassoul, kaolin (white, pink, yellow, red and rose), green illite, blue montmorillonite, Moroccan red, multani mitti; waxes selected from carnuba, beeswax, paraffin, synthetic waxes; rice bran; floral; hexagonal boron nitride ceramic powders; yucca shidigera powder; sodium ascorbyl phosphate; magnesium ascorbyl phosphate; hyaluronic acid; glass spheres, hollow glass spheres, ceramic spheres, silica spheres, alumina particles, polymer particles produced by grinding; poly(methyl methacrylate), polyethylene, ethylene/acrylate copolymer, Nylon-12, silicone resin, polyurethane polymers of average diameter ranging from about 0.4 to about 14 micrometers; hollow spheres of ethylene/methacrylate copolymer having an average diameters of from about 20 to about 32 micrometers; non-porous silica, porous silica, highly porous silica, and surface treated silica having an average diameter of about 2 to about 12 micrometers; titanium dioxide, having particle sizes ranging from about 20 nanometers to about 300 nanometers; zinc oxide ranging in particle diameter from about 20 to about 500 nanometers, alumina powder, silica-alumina powder, polymers which are prepared by spray drying from water or organic solvents, powders having particle sizes of from about 0.5 to about 30 micrometers, magnesium oxide powder or mixtures thereof.

38. The dermal patch of claim 30 wherein the first layer includes the active agent.

39. The dermal patch of claim 30 wherein the second layer includes the active agent.

40. The dermal patch of claim 38 or 39 wherein the first layer includes the water-dispersible or water-dissipatable film-forming polymer.

41. The dermal patch of claim 38 or 39 wherein the second layer includes the water-dispersible or water-dissipatable film-forming polymer.

42. The dermal patch of claim 30 wherein the first layer has a Tg of from about −5° C. to about −45° C. and the second layer has a Tg greater than about 5° C., and wherein the difference between the first layer Tg and the second layer Tg is at least about 15° C.

43. A method for making the dermal patch of claim 1 comprising the steps of:
a. applying a first layer to a first protective releasable substrate;
b. joining a second layer to the first layer; and
c. covering the second layer with a second protective releasable substrate;
wherein at least one of said first or second layers is a polymer matrix system having an active agent admixed therein, and wherein at least one layer comprises a water-dispersible or water-dissipatable polymer.

44. The method of claim 43 further comprising drying said first layer prior to joining the second layer to the first layer.

45. The composition of claim 2 wherein the second layer comprises a urethane polymer.

46. The method of claim 43, wherein the second layer comprises a urethane polymer.

47. The composition of claim 2, wherein the second layer comprises a carboxymethylcellulose acetate butyrate polymer.

48. The method of claim 43, wherein the second layer comprises a carboxymethylcellulose acetate butyrate polymer.
